(19) United States
(12) Patent Application Publication
(10) Pub. No.: US 2013/0065227 A1
(43) Pub. Date: Mar. 14, 2013

(54) METHODS OF INCREASING MACROPINOCYTOSIS IN CANCER CELLS

(71) Applicants: Paula J. Bates, Louisville, KY (US); Elsa Merit Reyes-Reyes, Louisville, KY (US)

(72) Inventors: Paula J. Bates, Louisville, KY (US); Elsa Merit Reyes-Reyes, Louisville, KY (US)

(21) Appl. No.: 13/580,863

(22) PCT Filed: Mar. 4, 2011
(86) PCT No.: PCT/US11/27124

§ 371 Date: Aug. 23, 2012

Related U.S. Application Data
(60) Provisional application No. 61/310,419, filed on Mar. 4, 2010.

(51) Int. Cl. C12N 5/09; C12Q 1/68; C12N 15/85; B82Y 5/00

(52) U.S. Cl. 435/6.1; 435/375; 435/455; 977/773; 977/915

(57) ABSTRACT

This disclosure describes methods of stimulating macropinocytosis in cancer cells.

### FEDERALLY SPONSORED RESEARCH OR DEVELOPMENT

This invention was made with government support under Grant No. CA 122383 awarded by the National Institutes of Health. The government has certain rights in the invention.

### TECHNICAL FIELD

This disclosure generally relates to methods of delivering therapeutic compounds to cancer cells.

### BACKGROUND

A number of therapies are currently used for treating cancer, including, for example, chemotherapy, radiation therapy, surgery, gene therapy, and bone marrow transplantation. Therapies that specifically target cancer cells and not non-malignant cells, however, are desirable.

### SUMMARY

This disclosure describes methods of stimulating macropinocytosis in cancer cells.

In one aspect, a method of stimulating macropinocytosis in cancer cells is provided. Such a method generally includes the steps of contacting the cancer cells with a G-rich nucleic acid that is capable of forming a quadruplex structure to thereby stimulate macropinocytosis in the cancer cells. In certain embodiment, the G-rich nucleic acid is between 10 and 50 nucleotides in length and is greater than 25% G nucleotides. In certain embodiments, the G-rich nucleic acid has a sequence shown in SEQ ID NO: 1. Representative cancer cells include, without limitation, prostate cancer, lung cancer, cervical cancer, breast cancer, colon cancer, pancreatic cancer, renal cell carcinoma, ovarian cancer, leukemia, lymphoma, melanoma, glioblastoma, neuroblastoma, sarcoma, and gastric cancer.

In another aspect, a method of delivering a therapeutic compound to cancer cells is provided. Such a method generally includes the steps of contacting the cancer cells with a G-rich nucleic acid that is capable of forming a quadruplex structure, and contacting the cancer cells with a therapeutic compound. According to this method, the therapeutic compound is taken up (i.e., endocytosed) by the cancer cells via macropinocytosis. In certain embodiment, the G-rich nucleic acid is between 10 and 50 nucleotides in length and is greater than 25% G nucleotides. In certain embodiments, the G-rich nucleic acid has a sequence shown in SEQ ID NO: 1. Representative cancer cells include, without limitation, prostate cancer, lung cancer, cervical cancer, breast cancer, colon cancer, pancreatic cancer, renal cell carcinoma, ovarian cancer, leukemia, lymphoma, melanoma, glioblastoma, neuroblastoma, sarcoma, and gastric cancer.

In certain embodiment, the therapeutic compound is a nucleic acid, a peptide, a small molecule, a drug, a chemical, an antibody or a nanoparticle. Representative nucleic acid, for therapeutic use, include antisense RNA, interfering RNA, immunostimulatory oligonucleotides, triple helix oligonucleotides, transcription factor decoy nucleic acids, aptamers, or plasmid DNA.

In still another aspect, a method of determining whether cancer cells are susceptible or refractory to the antiproliferative effects of a G-rich nucleic acid capable of forming a quadruplex structure is provided. Such a method generally includes the steps of contacting the cancer cells with the G-rich nucleic acid; and determining whether or not macropinocytosis is increased in the cancer cells contacted with the G-rich nucleic acid relative to cancer cells not contacted with the G-rich nucleic acid. Typically, an increase in macropinocytosis by the cancer cells contacted with the G-rich nucleic acid indicates that the cancer cells are susceptible to treatment with the G-rich nucleic acid, while the absence of an increase in macropinocytosis by the cancer cells contacted with the G-rich nucleic acid indicates that the cancer cells are refractory to treatment with the G-rich nucleic acid.

In certain embodiment, the G-rich nucleic acid is between 10 and 50 nucleotides in length and is greater than 25% G nucleotides. In certain embodiments, the G-rich nucleic acid has a sequence shown in SEQ ID NO: 1. Representative cancer cells include, without limitation, prostate cancer, lung cancer, cervical cancer, breast cancer, colon cancer, pancreatic cancer, renal cell carcinoma, ovarian cancer, leukemia, lymphoma, melanoma, glioblastoma, neuroblastoma, sarcoma, and gastric cancer. In some embodiments, the method is performed in vitro with cancer cells obtained from a patient diagnosed with cancer.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 are graphs showing that AS1411 cell internalization is an active process. Cells were plated 18 h before uptake analysis. After incubation as described, cells were washed with ice-cold PBS, incubated with 1 μg/ml 7-AAD, harvested and resuspended in 1% paraformaldehyde containing 2 μg/ml actinomycin D, then analyzed by flow cytometry. (Panel A) DU145 cells were incubated at 37° C. with fresh complete DMEM medium containing 10 μM FL-AS1411 (black line) or 10 μM FL-CRO (gray line) for the time indicated. (Panel B) DU145 cells were incubated for 2 h at 37° C. in complete or serum-free DMEM medium containing 10 μM FL-AS1411, or 10 μM FL-CRO, or no oligonucleotide. (Panel C) Various cell lines were incubated with 10 μM FL-AS1411 (black outline histogram), 10 μM FL-CRO (gray outline histogram) or without DNA (solid gray histogram) at 37° C. or 4° C. for 2 h. All experiments were repeated at least three times. Data are mean of three independent samples; bars, SE; *(p<0.05).

FIG. 2 are graphs showing that AS1411 is internalized by different endocytic mechanisms in DU145 cancer cells and in non-malignant Hs27 cells. DU145 or Hs27 cells were plated 18 h before uptake analysis. Cells were pre-treated as described with inhibitor (gray histogram) or the corresponding vehicle control (black histogram) before addition of 10 μM FL-AS1411 and incubation at 37° C. for 2 h. After incubation, cells were harvested and analyzed by flow cytometry. Pre-treatment conditions were at 37° C. with: (Panel A) 5 μM Cytochalasin D for 30 min; (Panel B) 80 μM Dynasore for 30 min; or (Panel C) 3 mM amiloride for 1 h. All experiments were repeated at least three times and representative data are shown. Solid gray histograms represent background autofluorescence of unstained cells.

FIG. 3 are photographs showing that AS1411 co-localizes with the macropinocytic marker, dextran. DU145 or Hs27 cells were incubated with the reagents indicated, then washed and fixed. Nuclei were stained with DAPI (blue). The distribution of markers was visualized by confocal microscopy and fluorescent images were overlaid to determine co-localization as indicated by the yellow color. (Panel A) 10 μM AS1411 labeled with Alexa Fluor 488 (green) and 0.2 mg/ml dextran-10K, macropinocytic marker, labeled with Alexa Fluor 594 (red) for 2 h at 37° C. (Panel B) Experiments similar to those in Panel A but using FL-CRO in place of FL-AS1411. (Panel C) Cells incubated with 5 μg/ml transferrin labeled with Alexa Fluor 488 (green) and 0.2 mg/ml dextran-10K labeled with Alexa Fluor 594 (red) for 30 min at 37° C. (Panel D) DU145 cells incubated with 5 μg/ml transferrin labeled with Alexa Fluor 594 (red) and 10 μM AS1411 labeled with Alexa Fluor 488 (green) for 30 min at 37° C. Scale bars, 10 μm.

FIG. 4 shows that AS1411 stimulates macropinocytosis in DU145 cancer cells but not in non-malignant Hs27 cells. (Panel A) DU145 cells were treated with 10 μM tAS1411 or 10 μM tCRO or no oligonucleotide in complete DMEM medium at 37° C. for the time indicated. After treatment, cell medium was changed for fresh complete medium containing 0.2 mg/ml dextran-10K labeled with Alexa Fluor 488, and cells were incubated for 30 min at 37° C. After incubation, cells were harvested and analyzed by flow cytometry to determine dextran uptake. (Panel B) The same experiment was performed using Hs27 cells. (Panel C) DU145 cells were treated with 10 μM tAS1411 or 10 μM tCRO or no oligonucleotide in complete DMEM medium at 37° C. for 48 h. After treatment, cell medium was changed for fresh complete medium containing 0.2 mg/ml dextran-10K tagged with Alexa Fluor 488, and cells were incubated for 30 min at 37° C. Then, cells were washed with cold PBS, added PBS containing 5 μg/ml PI and incubated on ice for 5 min. After washing with cold PBS, cells were fixed and the distribution of macropinocytic marker was visualized by confocal microscopy. The nucleus was stained with DAPI (blue). Scale bars, 10 μm. All experiments have been repeated at least three times. Data are mean of three independent samples; bars, SE; *(p<0.05).

FIG. 5 shows that AS1411 uptake after 2 h is not affected by knockdown of nucleolin expression. DU145 cells were transfected for 48 h without siRNA (mock, M), or with 30 nM of one of three different nucleolin siRNAs (NCL1, NCL2, NCL3) or a control siRNA (scramble, S), or contransfected with 10 nM of each nucleolin siRNAs (mix). (Panel A) Cells were lysed and total cell lyses were analyzed by Western blotting using the antibodies shown. (Panel B) Cell-surface proteins from intact transfected DU145 cells were labeled covalently with membrane-impermeable biotinylating agent. Cells were lysed, then biotinylated plasma membrane proteins were captured with streptavidin-agarose beads and analyzed by blotting with anti-nucleolin antibody (upper panel). After stripping, the membrane was reprobed with antibodies for a plasma membrane marker (anti-pan Cadherin) and a nuclear marker (anti-histone 3) to confirm the fractionation. Total lysate (Lys) was used as control. (Panel C) The medium of transfected cells was replaced by fresh complete DMEM medium containing no oligonucleotide (gray dashed histogram) or 10 μM FL-CRO (gray solid line histogram) or 10 μM FL-AS1411 (black solid line histogram) and incubated at 37° C. for 2 h. After incubation, cells were harvested and analyzed by flow cytometry.

FIG. 6 are graphs showing that nucleolin regulates AS1411-induced stimulation of macropinocytosis. DU145 cells were untreated (no transfection) or transfected, without siRNA (mock), or with 30 nM of one of three different nucleolin siRNAs (NCL1, NCL2, NCL3) or a control siRNA (scramble). 48 h after transfection, cells were incubated with 10 μM tAS1411, 10 μM tCRO, or no oligonucleotide in complete DMEM medium at 37° C. for 24 h. (Panel A) After treatment, cell medium was changed for fresh complete medium containing 0.2 mg/ml dextran-10K labeled with Alexa Fluor 488 (green) and incubated for 30 min at 37° C. (Panel B) After treatment, some cells were washed, fresh complete medium containing 10 μM FL-AS1411 added and incubated for 2 h at 37° C. After incubation, cells were harvested and analyzed by flow cytometry. Mean fluorescence was normalized to “not transfected” control (Panel A) or to no pre-treatment control (Panel B). All experiments have been repeated at least three times. Data are the mean of three independent samples; bars, SE; *(p<0.05).

FIG. 7 are graphs showing the results of comparative experiments. (Panel A) Cells were plated in 96-well plates at low density (1,000 cells per well) and incubated 18 hrs at 37° C. to allow adherence. Cells were treated by addition of different concentrations of AS1411 (obtained from Antisoma), AS1411 (obtained from Invitrogen), tAS1411, FL-AS1411, CRO, or tCRO directly to the medium, and proliferation was measured using a 3-(4,5-dimethylthiazol-2-yl)-2,5-diphenyltetrazolium bromide assay as described previously (Bates et al., 1999, J. Biol. Chem., 274:26369-77). Points represent mean of triplicate samples with SE. (Panel B) 10 μM FL-AS1411 or 10 μM FL-CRO were added to DU145 cells plated on 6-well plates. After incubation at 37° C. for 2 hrs, cells were washed twice with ice-cold PBS and harvested by trypsin treatment. Some cells were washed twice with ice-cold PBS containing dextran sulfate (100 mg/ml) or trypan blue (250 mg/ml, pH 4.4). Cells were resuspended in ice-cold PBS and immediately analyzed by flow cytometry.

FIG. 8 is a graph indicating that uptake of GROs is receptor independent. DU145 cells were plated 18 hr before uptake analysis. Cell medium was changed with fresh complete DMEM medium containing different concentrations of FL-AS1411 (black line) or FL-CRO (gray line), and incubated at 37° C. for 2 hrs. After incubation, cells were harvested and analyzed by flow cytometry.

FIG. 9 are graphs showing the effects of inhibitors on various endocytic pathways. DU145 or Hs27 cells were plated 18 hr before uptake analysis. (Panel A) DU145 cells were pre-treated with dynamin inhibitor, 80 mM Dynasore (black histogram) or DMSO (gray histogram) for 30 min at 37° C. After pre-treatment, cells were treated with 5 mg/ml transferrin conjugated with Alexa Fluor 488 for 30 min at 37° C. (Panel B) DU145 and Hs27 cells were pre-treated with 3 mM amiloride (black histogram) or vehicle (serum-free medium, gray histogram) for 1 h at 37° C. After pre-treatment, FL-CRO was added at a concentration of 10 μM and incubated at 37° C. for 2 h. After incubation, cells were harvested and analyzed by FACS. Gray solid histogram represents unstained cells.

FIG. 10 are graphs showing the stimulation of macropinocytosis by GROs. Breast carcinoma cells (MDA-MB-231, MCF-7) or non-malignant breast epithelial cells (MCF10A) cells were plated 18 hr before treatment with 10 μM tAS1411 or 10 μM tCRO or water in complete DMEM medium at 37° C. for 48 hr or 72 hr. After treatment, cell medium was changed for fresh complete medium containing 0.2 mg/ml dextran-10K labeled with Alexa Fluor 488 and cells were incubated for 30 min at 37° C. Cells were then incubated on ice with 1 mg/ml PI in PBS, harvested, and analyzed by flow cytometry. Uptake was normalized to no pre-treatment controls and bars show the mean and SE of three independent experiments.

FIG. 11 is a graph showing a comparison between AS1411 and tAS1411 in the ability to stimulate macropinocytosis. DU145 cells were plated 18 hr before treatment with different concentrations (0, 5, 10, or 15 μM) of AS1411, tAS1411 or tCRO in complete DMEM medium at 37° C. for 48 h. After treatment, cell medium was changed for fresh complete medium containing 0.2 mg/ml dextran-10K labeled with Alexa Fluor 488, and cells were incubated for 30 min at 37° C. After incubation, cells were incubated on ice with 1 mg/ml PI in PBS, harvested, fixed and immediately analyzed by flow cytometry.

FIG. 12 are graphs showing the effectiveness of pre-treatment of cells with a GRO. DU145 or Hs27 cells were treated with or without 10 μM tAS1411 in complete DMEM medium at 37° C. for 24 h. After treatment, cells were washed, fresh complete medium containing 10 μM FL-AS1411 was added, and cells were incubated for a further 2 h at 37° C. After incubation, cells were incubated on ice with 1 mg/ml PI in PBS, harvested, fixed and immediately analyzed by flow cytometry.

FIG. 13 are graphs showing the effects of anti-nucleolin antibodies. (Panel A) DU145 cells were harvested and incubated with different anti-nucleolin antibody clones: MS3 (10 or 40 mg/ml), E42 (10 mg/ml) or D3 (10 or 40 mg/ml), or non-immune isotype control mouse IgG (10 or 40 μg/ml), followed by Alexa Fluor 488-conjugated anti-mouse IgG-Fc F(ab)2, and analyzed by flow cytometry. (Panel B) DU145 or Hs27 cells were plated 18 hr before pre-treatment with anti-nucleolin antibody D3 (10 or 20 mg/ml), or isotype control mouse IgG (10 or 20 μg/ml) for 15 min at 4° C. After pre-treatment, cells were treated with 10 μM FL-AS1411 or 10 μM FL-CRO for 2 h at 37° C. After incubation, cells were incubated on ice with 1 mg/ml PI in PBS, harvested, fixed and immediately analyzed by flow cytometry.

FIG. 14 shows induction of non-apoptotic cell death by AS1411. (A) Trypan blue staining of U937 leukemia cells showing percentage of dead cells (trypan blue positive) over time. (B) U937 cells were untreated (Un) or treated with 1 μM AS1411 (1411) or control oligonucleotide (Ctrl) for 72 h. DNA was extracted, electrophoresed on an agarose gel and stained with ethidium bromide to probe DNA laddering. As a positive control, gels were treated with UV irradiation to induce apoptosis as previously described. (C) Electron micrographs showing ultrastructure of U937 cells that had been treated for 72 h with AS1411 (two fields are shown) compared to control. (D) U937 cells treated as described for panel B, followed by protein extraction and immunoblotting (IB) to detect PARP cleavage and Caspase-3 activation. (E) DU145 prostate cancer cells were incubated with 10 μM of AS1411 for 24 h, or irradiated with UV (300 J/m² with UV Stratalinker 2400, Strategene), then cultured in fresh medium for 6 h. Assessment of cell death was carried out by flow cytometry to detect Annexin V-FITC binding and PI staining.

FIG. 15 shows a graph indicating that the autophagy inhibitor, 3-MA, does not block AS1411 activity. DU145 cells were incubated for 4 days in the presence of 10 μM AS1411 with 3-MA at the concentration indicated, and cell number was assessed by MTT assay. 3-MA has some toxicity by itself and the effect of AS1411 was additive.

FIG. 16 shows a spheroid culture of DU145 cells and inhibition by AS1411. DU145 CD24^{lo}/CD44^{hi} cells were sorted by FACS, plated for sphere culture, and treated with or without 10 μM AS1411. Media was changed and drug replenished weekly. Plates were monitored for dissolution of spheres. Once dissolution was observed, serial photographs were taken of each well and the total number of spheres counted.

FIG. 17 shows graphs demonstrating the dependence of AS1411-stimulated MP or anti-proliferative activity on EGFR, Ras, Rac, PI3K, and Nucleolin. Except where stated, experiments used DU145 cancer cells treated with 10 μM AS1411 or inactive control oligonucleotide. (A) NIH-3T3 fibroblasts were stably transfected with pZIP empty vector or pZIP-H-Ras (G12V). Cells were treated for 4 days as indicated and assessed by MTT assay. (B) DU145 cells were treated as described and 34 μg of cell lysate was used to measure Rac activation using G-Lisa Rac Activation Assay Biochem (Cytoskeleton #BK125). (C) After pre-treatment as described, cells were incubated with the indicated inhibitor at appropriate concentrations, MP was measured by flow cytometry using dextran 10K-Alexa488 with gating to exclude PI+ cells. (D) Reyes-Reyes et al., 2010, Cancer Res., 70:8617-8629. (E,F) Experiments as in panel C, except using Rac or PI3K inhibitors at the concentrations indicated.

FIG. 18 is a graph showing the uptake of siRNA in the presence of AS1411.

### DETAILED DESCRIPTION

This document discloses that G-rich nucleic acids capable of forming quadruplex structures stimulate macropinocytosis in cancer cells but not in non-malignant cells. Macropinocytosis is a type of endocytosis that is distinguishable from other endocytic pathways. Unlike both receptor-mediated endocytosis and phagocytosis, macropinocytosis is not regulated through direct actions of cargo/receptor molecules coordinating the activity and recruitment of specific effector molecules of particular sites at the plasma membrane.

Macropinosomes are derived from actin-rich extensions of the plasma membrane, referred to as ruffles. Membrane ruffling occurs due to actin polymerization near the plasma membrane. As the newly formed actin branch grows, the plasma membrane is forced out, extending the membrane into a ruffle. Macropinosomes are formed when these ruffles fuse back with the plasma membrane and encapsulate a large volume of extracellular fluid in the process. Macropinosome formation can be inhibited with amiloride, an ion exchange inhibitor, or derivatives thereof, with no detectable effect on the other endocytic pathways. Therefore, in concert with the morphological description, suppression with amiloride (and, optionally, elevation in response to growth factor stimulation) is used to define macropinocytosis and distinguish macropinocytosis from other types of endocytosis.

As demonstrated herein, G-rich nucleic acids stimulate micropinocytosis in cancer cells but not in non-malignant cells. G-rich nucleic acids have been shown to adopt intermolecular or intramolecular quadruplex structures that are stabilized by the presence of G-quartets. G-quartets are square planar arrangements of four hydrogen-bonded guanines that are stabilized by monovalent cations. See, for example, Dapic et al. (2003, Nuc. Acids Res., 31:2097-107). Significantly, G-rich nucleic acids have been shown to exhibit antiproliferative effects on a number of different types of cancer cells. See, for example, Bates et al., 2009, Exp. Mol. Path., 86:151-64.

As used herein, G-rich nucleic acids refer to nucleic acids (e.g., DNA or RNA) that contain a guanine content that is sufficient for formation of quadruplex structures. Although there is not a particular guanine content required for quadruplex formation, G-rich oligonucleotides typically are greater than 25% guanine. G-rich nucleic acids include oligonucleotides between, for example, 12 nucleotides and 50 nucleotides in length (e.g., 15, 18, 20, 21, 22, 23, 24, 25, 26, 27, 28, 29, 30, 33, 35, 38, 40, 42, 45 or 48 nucleotides in length). G-rich nucleic acids also include nucleic acids greater than 50 nucleotides in length including, for example, nucleic acids that are 100 nucleotides or more in length, 250 nucleotides or more in length, 500 nucleotides or more in length, 1000 nucleotides (i.e., 1 kilobase (Kb)) or more in length, 2 Kb or more in length, 3 Kb or more in length, 4 Kb or more in length, or 5 Kb or more in length. G-rich nucleic acids can have modifications to, for example, the backbone (e.g., peptide nucleic acid (PNA), or phosphorothioation), one or more of the bases (e.g., methylation, glycosylation, thiol-modification, or a label (e.g., fluorescence or a radiolabel)), or the 3′ or 5′ end (e.g., a label), provided that the modification does not disrupt the ability of the G-rich nucleic acid to form quadruplex structures.

Because macropinocytosis in cancer cells is stimulated by G-rich nucleic acids, this phenomenon can be utilized to deliver one or more therapeutic compounds to the cancer cells. A therapeutic compound that can be delivered to cancer cells includes, without limitation, nucleic acids, peptides, small molecules, drugs, chemicals, antibodies or nanoparticles. Since non-malignant cells still undergo macropinocytosis to a limited degree, the specificity afforded by using therapeutic compounds such as nucleic acids may be preferred. Representative nucleic acids can be, for example, antisense RNA, interfering RNA (e.g., siRNA), immunostimulatory oligonucleotides (e.g., CpG motif-containing oligonucleotides), triple helix oligonucleotides, transcription factor decoy nucleic acids, aptamers, or plasmid DNA. In addition, a therapeutic compound such as a nucleic acid may be linked to or contiguous with the G-rich nucleic acid.

One or more G-rich nucleic acids and/or one or more therapeutic compounds can be delivered to cancer cells via any number of means. For example, one or more G-rich nucleic acids and/or one or more therapeutic compounds can be delivered to cancer cells via direct injection (e.g., into a solid tumor), intravenous administration, intraperitoneal administration, subcutaneous administration, oral administration or administration by inhalation. The one or more G-rich nucleic acids can be delivered to the cancer cells prior to delivery of the one or more therapeutic compounds (e.g., to allow the induction of macropinocytosis to occur), or the one or more G-rich nucleic acids and the one or more therapeutic compounds can be delivered to cancer cells simultaneously or essentially simultaneously. If delivered simultaneously, the one or more G-rich nucleic acids and the one or more therapeutic compounds can be delivered via a single composition or via separate compositions.

G-rich nucleic acids have been shown herein to stimulate macropinocytosis in prostate cancer, lung cancer, cervical cancer and breast cancer. Since, in addition to prostate cancer, lung cancer, cervical cancer and breast cancer, G-rich nucleic acids have been shown to exhibit antiproliferative effects against colon cancer, pancreatic cancer, renal cell carcinoma, ovarian cancer, leukemia and lymphoma, melanoma, glioblastoma, neuroblastoma, sarcoma, and gastric cancer, it is expected that G-rich nucleic acids would stimulate macropinocytosis in these cancers as well.

Whether or not macropinocytosis is stimulated can be used as a marker to determine whether cancer cells are susceptible or refractory to the antiproliferative effects of a G-rich nucleic acid. For example, cancer cells treated with a G-rich nucleic acid can be evaluated to determine whether or not there is an increase in macropinocytosis. An increase in macropinocytosis in cancer cells treated with a G-rich nucleic acid generally indicates cancer cells that are susceptible to the G-rich nucleic acid, while the lack of an increase indicated cancer cells that are refractory to the G-rich nucleic acid.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Materials

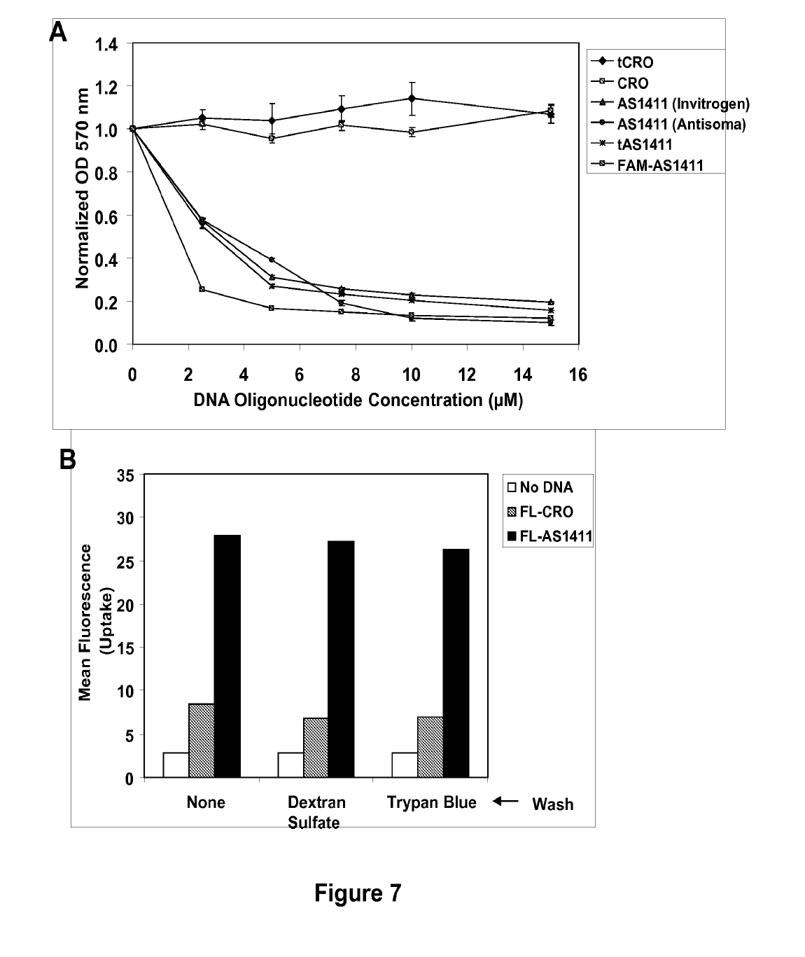

Oligodeoxynucleotides were purchased from Invitrogen (Carlsbad, Calif.). Sequences used for this study include: AS1411, 5′-d(GGT GGT GGT GGT TGT GGT GGT GGT GG) (SEQ ID NO:1); FL-AS1411 (fluorophore-labeled AS1411), 5′-Fluor-d(TTT GGT GGT GGT GGT TGT GGT GGT GGT GG) (SEQ ID NO:2), where Fluor is either 5-Carboxyfluorescein (FAM, used for flow cytometry studies) or Alexa Fluor 488 (used for confocal microscopy); tAS1411, 5′-d(TTT GGT GGT GGT GGT TGT GGT GGT GGT GG) (SEQ ID NO:3); FL-CRO, 5′-Fluor-d(TTT CCT CCT CCT CCT TCT CCT CCT CCT CC) (SEQ ID NO:4); CRO, 5′-d(CCT CCT CCT CCT TCT CCT CCT CCT CC) (SEQ ID NO:5); and tCRO, 5′-d(TTT CCT CCT CCT CCT TCT CCT CCT CCT CC) (SEQ ID NO:6). Unmodified oligonucleotides were purchased in the desalted form, whereas fluorescently labeled sequences were HPLC purified. The 29-mer sequences were used for some experiments because quenching of the fluorophore occurred when it was located adjacent at the 5′-terminal base of the AS1411 sequence, so a spacer consisting of 3 thymidines was added. The antiproliferative activities of 29-mer sequences, with and without the fluorophore, were comparable to the synthesized 26-mer AS1411 sequence, as well as to AS1411 obtained from Antisoma (see FIG. 7). The dextran, 10,000 MW, anionic fixable (dextran-10K) and transferrin (Tf) conjugated with Alexa Fluor 488 or Alexa Fluor 594 were purchased from Invitrogen. Anti-rabbit and anti-mouse antibodies linked to horseradish peroxidase, anti-histone 3 rabbit polyclonal and anti-pan cadherin (C19) goat polyclonal antibodies were purchased from Santa Cruz Biotech (Santa Cruz, Calif.). Anti-nucleolin monoclonal antibodies were obtained from Stressgen (4E2) and Santa Cruz Biotech (MS-3). The anti-nucleolin mAb (D3) was a generous gift from Dr. Jau-Shyong Deng, University of Pittsburgh School of Medicine. Cytochalasin D (actin polymerization inhibitor), dynasore (dynamin inhibitor), and amiloride (macropinocytosis inhibitor) were from Calbiochem (San Diego, Calif.). Triton X-100 was purchased from Sigma (Saint Louis, Mo.), paraformaldehyde was from Electron Microscopy Sciences (Hatfield, Pa.), and dimethylsulfoxide (DMSO) was from the American Type Culture Collection (ATCC, Manassas, Va.).

### Example 2

### Cell Culture and Treatment

All cells were obtained from the American Type Culture Collection (ATCC) and grown in a humidified incubator maintained at 37° C. with 5% CO₂. Hs27 (non-malignant human foreskin fibroblasts), DU145 (hormone-refractory prostate cancer), A549 (non-small cell lung cancer), HeLa (cervical adenocarcinoma), MCF-7 (hormone-dependent breast cancer) and MDA-MB-231 (hormone-independent breast cancer) cells were grown in DMEM supplemented with 10% fetal bovine serum (FBS; Life Technologies), 62.5 μg/mL penicillin and 100 μg/mL streptomycin (Hyclone Laboratories, Logan, Utah). MCF-10A cells (immortalized human breast epithelial cells) were grown in MEBM supplemented with all the components of MEGM bullet kit (Lonza, Allendale, N.J., Catalog No. 3150) except for the GA-1000. Cells were plated at 50% confluence and incubated 18 h to allow adherence, and then the medium was changed for fresh supplemented medium and treated by addition of oligodeoxynucleotides directly to the culture medium to give the final concentration indicated in the Description of the Drawings. Dynasore and cytochalasin D were dissolved in DMSO. Amiloride was dissolved in serum-free medium. Cells were pre-treated with inhibitors in serum-free medium for either 30 min (cytochalasin D) or 60 min (dynasore and amiloride). Cells for biochemical analyses were lysed in lysis buffer (150 mM NaCl, 2 mM EDTA, 50 mM Tris-HCl, 0.25% deoxycholic acid, 1% IGEPAL® CA-630, pH 7.5) containing protease and phosphatase inhibitor cocktails (Calbiochem, Catalogs No. 539134 and 544625) for 20 min at 4° C. and then cleared by centrifugation at 16,000×g for 10 min at 4° C. All protein concentrations were determined using the BCA assay (Pierce, Rockford, Ill.).

### Example 3

### Flow Cytometric Assays

To analyze uptake of the oligodeoxynucleotides or dextran-10K (macropinocytic marker) by flow cytometry, 2×10⁵ cells in fresh supplemented culture medium (2.5 ml) were plated into 6-well plates for 18 h. After complete adhesion, the cells were incubated with 5′-FAM tagged oligodeoxynucleotides or Alexa Fluor 488 tagged dextran-10K and incubated as indicated in the Description of the Drawings. Cells were washed once with ice-cold PBS, incubated with 1 μg/ml 7-amino-actinomycin D (7-AAD) for 5 min on ice or 1 μg/ml propidium iodide (PI), and washed twice with ice-cold PBS. Cells were then treated with 0.01% trypsin/0.5 mM EDTA (300 μl) for 3 min prior addition 3 ml supplemented culture medium. The cells were then centrifuged and resuspended in 0.5 ml of 1% paraformaldehyde for analysis by flow cytometry using a FACScalibur cytometer (BD Biosciences, Mountain View, Calif.).

### Example 4

### Immunofluorescence Microscopy

Cells (4×10⁴) in fresh supplemented culture medium were plated on 18 mm diameter glass cover slips for 18 h. The media was removed and replaced with serum-free medium containing 10 μM oligodeoxynucleotide, dextran-10K, or transferrin and incubated as describe in the Description of the Drawings. After incubation, cells were washed 3 times with ice-cold PBS, fixed in 4% paraformaldehyde in PBS for 30 min at room temperature, and washed three times with PBS. After washing, the cover slips were mounted on glass slides with ProLong Antifade (Molecular Probes) according to the manufacturer's directions to inhibit photobleaching. Immunofluorescence was documented with an LSM 510 inverted confocal laser-scanning microscope (Carl Zeiss, Oberkochen, Germany) equipped with an Omnichrome argon-krypton laser. Images were obtained with a Zeiss Plan-Apo 63× oil immersion objective (1.4 NA).

### Example 5

### Biotinylation and Purification of Cell-Surface Proteins

Plated cells were washed three times with ice-cold PBS and added freshly prepared solution of 0.5 mg/ml of a cell-impermeable biotinylating agent (sulfo-NHS-biotin, Pierce, Rockford, Ill.) in PBS. After incubation for 30 min at 4° C., cell were washed once with ice-cold TBS (50 mM Tris-HCl, 150 mM NaCl, pH 7.5), incubated with ice-cold supplemented culture media for 10 min at 4° C., and then washed twice with TBS. Biotinylated proteins were precipitated by incubating with high capacity Neutravidin agarose (Pierce) for 2 h at 4° C. with gentle agitation, and then washed with ice-cold lysis buffer.

### Example 6

### RNA Interference

The nucleolin siRNA sequences were: 5′-GGU CGU CAU ACC UCA GAA Gtt/5′-CUU CUG AGG UAU GAC GAC Ctc (NCL1) (SEQ ID NO:7); 5′-GGC AAA GCA UUG GUA GCA Att/5′-UUG CAU CCA AUG CUU UGC Ctc (NCL2) (SEQ ID NO:8); and 5′-CGG UGA AAU UGA UGG AAA Utt/5′-AUU UCC AUC AAU UUC ACC Gtc (NCL3) (SEQ ID NO:9), targeted to non-conserved regions of the nucleolin open reading frame (GenBank Accession No. NM_{—}005381). BLAST analysis showed no homology of the siRNA sequences to any other sequence in the Human Genome Database. The siRNA nucleotides were chemically synthesized and annealed by Ambion Inc. (Austin, Tex.). Nucleolin siRNAs (30 nM) were transfected in DU145 cells using Lipofectamine 2000 (Invitrogen), according to the manufacturer's directions. The scrambled siRNA used as a negative control was obtained from Ambion.

### Example 7

### Immunoblotting

Samples were resolved by 10% SDS-Tris polyacrylamide gel electrophoresis and then electrotransferred onto polyvinylidine fluoride (PVDF) membranes (Millipore, Bedford, Mass.) in Tris-glycine buffer containing 20% methanol. Proteins were detected by immunoblotting as described (Reyes-Reyes et al., 2006, Exp. Cell Res., 312:4056-69). In some cases, PVDF membranes were stripped of bound antibodies using 62.5 mM Tris-HCl, pH 6.7, 100 mM 2-mercaptoethanol, 2% SDS for 30 min at 60° C. and then reprobed as described in the Description of the Drawings.

### Example 8

### Densitometry and Statistical Analysis

In some experiments, densitometry was used to measure band intensities by scanning autoradiographic films and using UN-SCAN-IT gel software (Silk Scientific Corporation). Band intensities were normalized as indicated in the Description of the Drawings. The statistical comparisons between AS1411-treated and control groups were carried out using Student's t test, and differences are indicated as *(p<0.05).

### Example 9

### Uptake of FL-AS1411 Occurs Through an Active Uptake Process

To first identify suitable conditions to study the mechanism of AS1411 uptake, the timing and serum-dependence of uptake was analyzed in DU145 prostate cancer cells, which are sensitive to AS1411. Uptake of FL-AS1411, a fluorescently labeled version of the active aptamer, and FL-CRO, a fluorescently labeled control oligonucleotide with no antiproliferative activity, was examined by flow cytometry with gating to exclude non-viable cells. Cell-associated fluorescence was not influenced by washing the cells with dextran sulfate to remove the extracellular fluorophore-labeled DNA or by adding trypan blue to quench external fluorescent signals prior to flow cytometry, ruling out the possibility that fluorophore-labeled DNA fluorescent signal is emanating from cell surface (see FIG. 7B).

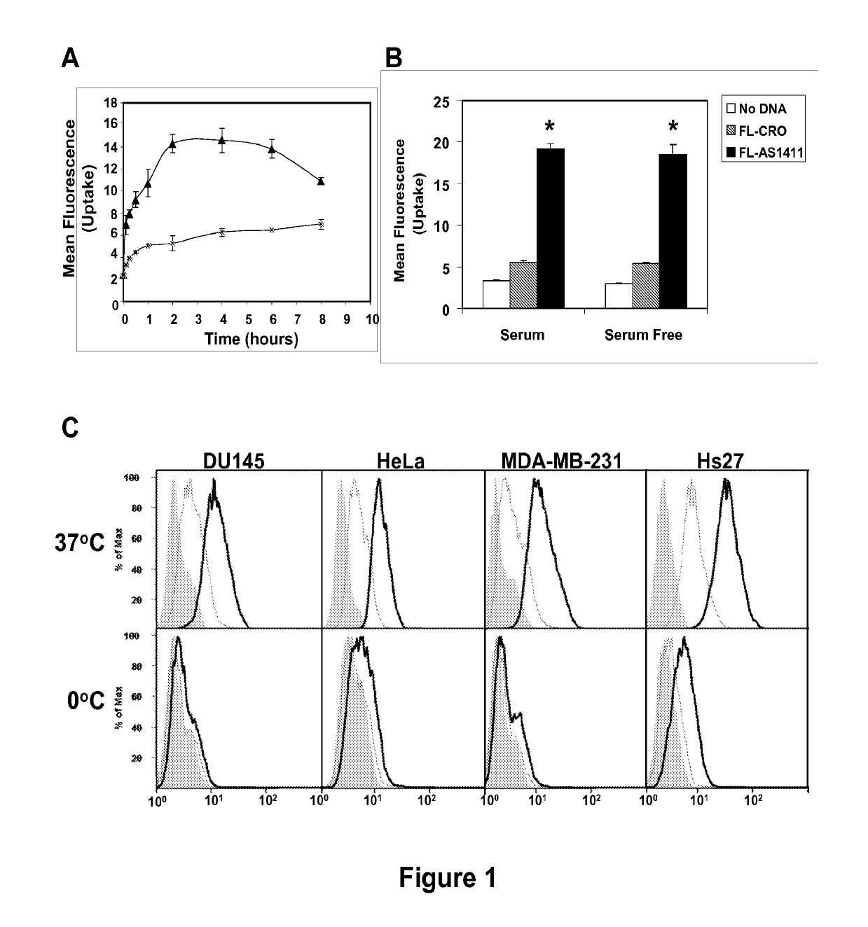

FL-AS1411 uptake was detected as early as 5 min, with maximum uptake between 2 h and 4 h, and decreasing after 8 h under these conditions (FIG. 1A). FL-CRO uptake was consistently much lower than FL-AS1411 and followed different kinetics. As shown in FIG. 1B, uptake of FL-AS1411 was independent of the presence of serum in the medium.

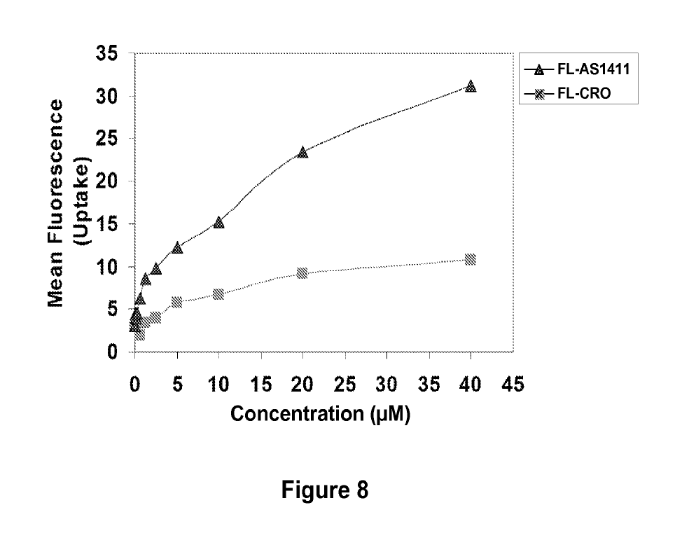

To determine whether AS1411 uptake occurs through an active uptake process, the temperature-dependence of AS1411 uptake in cancer cells (DU145, HeLa, MDA-MB-231) and non-malignant Hs27 skin fibroblasts was evaluated using flow cytometry. In all cell types, the uptake of FL-AS1411 and FL-CRO showed strong temperature dependence. However, in contrast to the original hypothesis, Hs27 cells appeared to have a higher uptake of AS1411 than any of the cancer cells analyzed (FIG. 1C). Next, it was tested whether FL-AS1411 uptake was concentration-dependent. Dose-response experiments in DU145 cells showed that AS1411 did not appear to have saturable uptake and presented an almost linear increase between 1.25 μM and 40 μM (FIG. 8), suggesting that uptake is receptor independent. Higher concentrations of AS1411 resulted in apparent cytotoxicity, even at this early time point.

### Example 10

### FL-AS1411 Uptake Occurs Through Different Endocytic Mechanisms in Cancer Cells and in Non-Malignant Cells

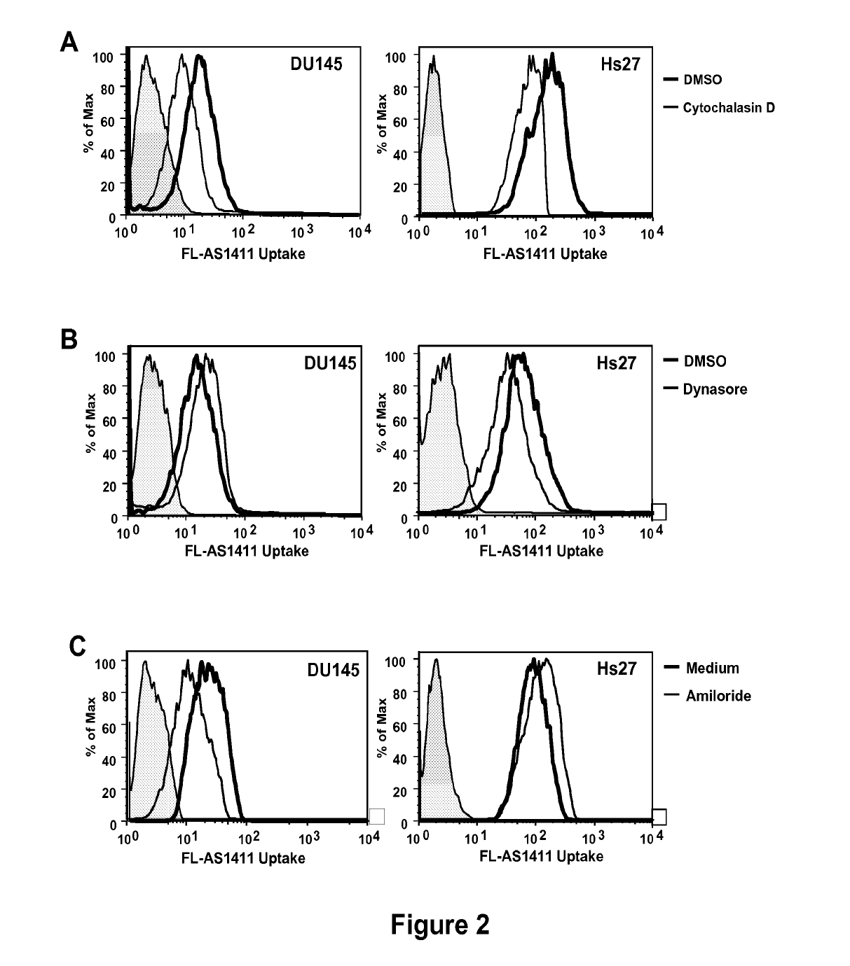

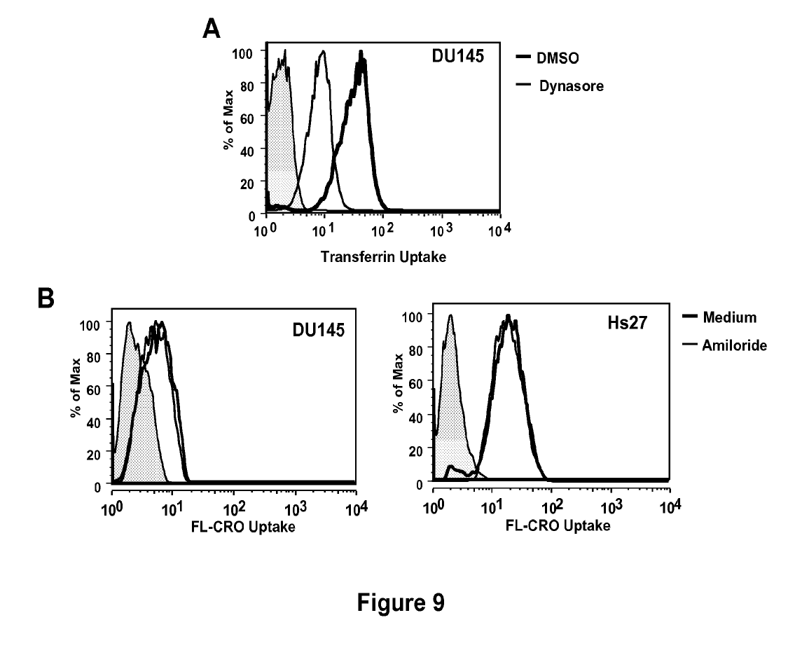

To confirm that uptake of AS1411 occurs by endocytosis, the involvement of the actin cytoskeleton, which has been implicated in regulating endocytic pathways, was evaluated. To this end, DU145 and Hs27 cells were pre-treated with an actin polymerization inhibitor. 5 μM cytochalasin D, and assessed for FL-AS1411 uptake by flow cytometry. Cytocholasin D-treated cells showed a decrease in FL-AS1411 uptake compared with the untreated cells (FIG. 2A). These data strongly suggest that AS1411 uptake occurs through endocytosis. In recent years, the vast complexity of endocytosis has been realized and recognized pathways now include caveolae-mediated endocytosis, clathrin- and caveolae-independent endocytosis, and macropinocytosis (Doherty et al., 2009, Anna. Rev. Biochem., 78:857-902), in addition to classical clathrin-mediated endocytosis. The GTPase dynamin is required for clathrin- and caveolae-mediated endocytosis and some clathrin and caveolae-independent endocytic pathways (Doherty et al., supra). Therefore, the effect of dynasore, a potent inhibitor of dynamin function (Macia et al., 2006, Dev. Cell, 10:839-50), on FL-AS1411 uptake in cancer (DU145) and nonmalignant (Hs27) cells (FIG. 2B) was investigated. Pre-treatment of Hs27 cells with 80 μM dynasore decreased the uptake of AS1411 (FIG. 2B). In contrast, pre-treatment of DU145 cells slightly increased the uptake of FL-AS1411. To rule out the possibility that DU145 cells were unresponsive to dynasore treatment, it was demonstrated that uptake of transferrin, a well-established ligand of clathrin-dependent endocytosis, was inhibited in DU145 cells pre-treated with dynasore, (FIG. 9A). These results suggest that AS1411 may be taken up by different endocytotic pathways in the cancer cells compared to the non-cancer cells, possibly following a predominantly clathrin or caveolae-dependent route of entry in Hs27 cells, but not in DU145 cells.

### Example 11

### Macropinocytosis is the Predominant Mechanism of Uptake for AS1411 in Cancer Cells

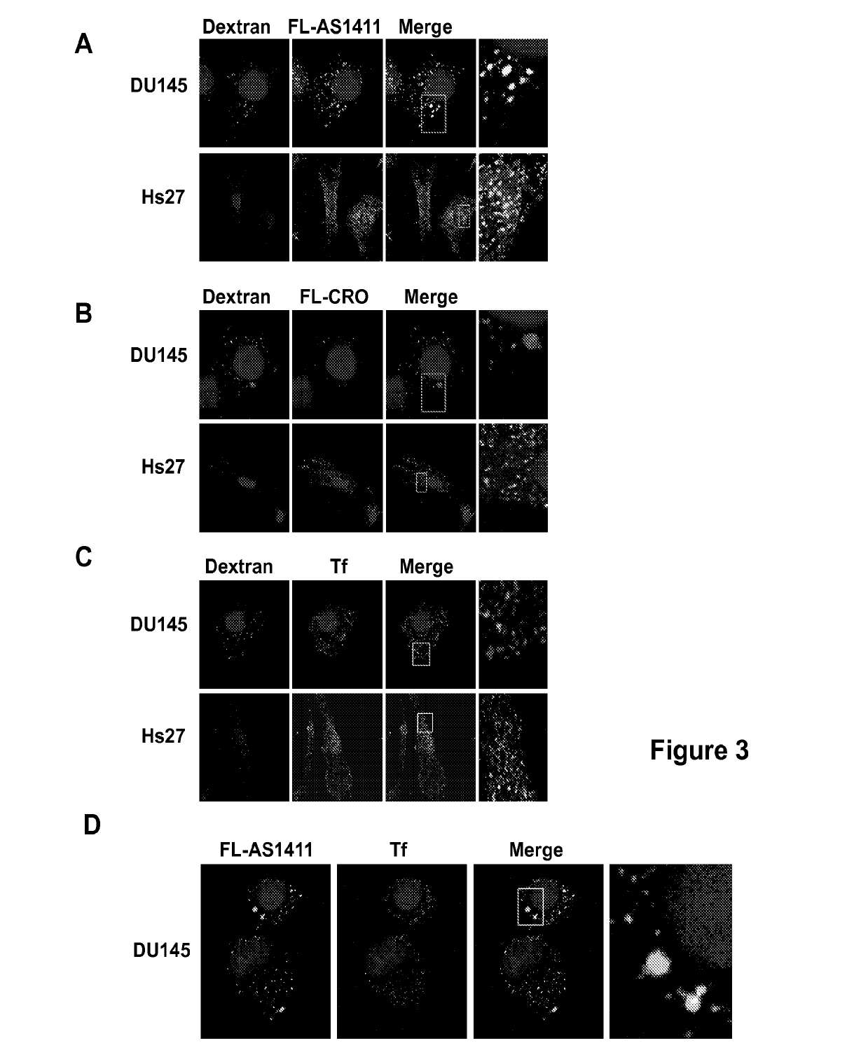

Recent work has showed that internalization of DNA plasmids or oligonucleotides can be mediated through macropinocytosis (Basner-Tschakarjan et al., 2004, Gene Ther., 11:765-74; Fumoto et al., 2009, Mol. Pharm., 6:1170-9; Wittrup et al., 2007, J. Biol. Chem., 282:27897-904), an actin-driven, ligand-independent mechanism in which cells “gulp” the surrounding medium and any macromolecules it contains. This endocytic mechanism has been shown to be sensitive to amiloride, a specific inhibitor of Na+/H exchange (West et al., 1989, J. Cell. Biol., 109:2731-9) and, therefore, the effect of this inhibitor on FL-AS1411 uptake was tested. It was found that amiloride pre-treatment caused a reduction in FL-AS1411 uptake only in DU145 cancer cells, but not in the non-malignant Hs27 cells (FIG. 2C). There was little effect of amiloride treatment on uptake of FL-CRO in either DU145 cancer cells or non-malignant Hs27 cells (FIG. 9B). Amiloride treatment also affected the AS1411 uptake in other cancer cells including MCF7 and MDA-MB-231 cells. These data strongly suggest that macropinocytosis could be responsible for the internalization of AS1411 in cancer cells. Confocal microscopy studies showed that FL-AS1411 was localized in confined structures in the cytoplasm of cancer and non-malignant cells (FIG. 3). As expected, uptake of FL-CRO was much lower than FL-AS1411, but it was similarly localized in cytopasmic foci. Interestingly, these studies showed that macropinocytosis (indicated by dextran uptake) is much more active in DU145 cancer cells than in the non-malignant Hs27 cells (FIG. 3). Moreover, internalized FL-AS1411 was strongly co-localized with the macropinocytic marker, dextran, in DU145 cells (FIG. 3A), but not in Hs27 cells (FIG. 3B). These studies also confirmed the previous results that there was higher overall uptake of FL-AS1411 in Hs27 cells than in DU145 cells. Notably, no FL-AS1411 was observed in the nuclear region in these studies, and the nuclear and diffuse cytoplasmic localization of AS1411 observed in some earlier studies may have been an artifact produced by cell permeabilization or cellular death.

Further experiments were performed to confirm the identity of the vesicles containing FL-AS1411. Macropinosomes lack a clathrin coat and can be distinguished from endosomes by their comparative inability to concentrate receptors (Thomas et al., 2004, PLoS Biol., 2:1363-80). Therefore, cells were incubated with dextran-Alexa Fluor 488 together with a ligand for the transferrin receptor, transferrrin-Alexa Fluor 594 (FIG. 3C) or FL-AS1411 (labeled with Alexa 488) together with transferrrin-Alexa Fluor 594 (FIG. 3D). It was observed that, as expected, transferrin and dextran were mainly localized in distinct non-overlapping vesicles in DU145 and Hs27 cells (FIG. 3C). Transferrin and AS1411 also showed non-overlapping vesicles in DU145 cells (FIG. 3D) and the large vesicles containing FL-AS1411 or dextran were distinct from the much smaller endosomes that sequestered transferrin, suggesting that they are not internalized together. These data support the hypothesis that the endocytic process that regulates the internalization of AS1411 in cancer cells is macropinocytosis.

### Example 12

### A51411 Hyperstimulates Macropinocytosis in Cancer Cells

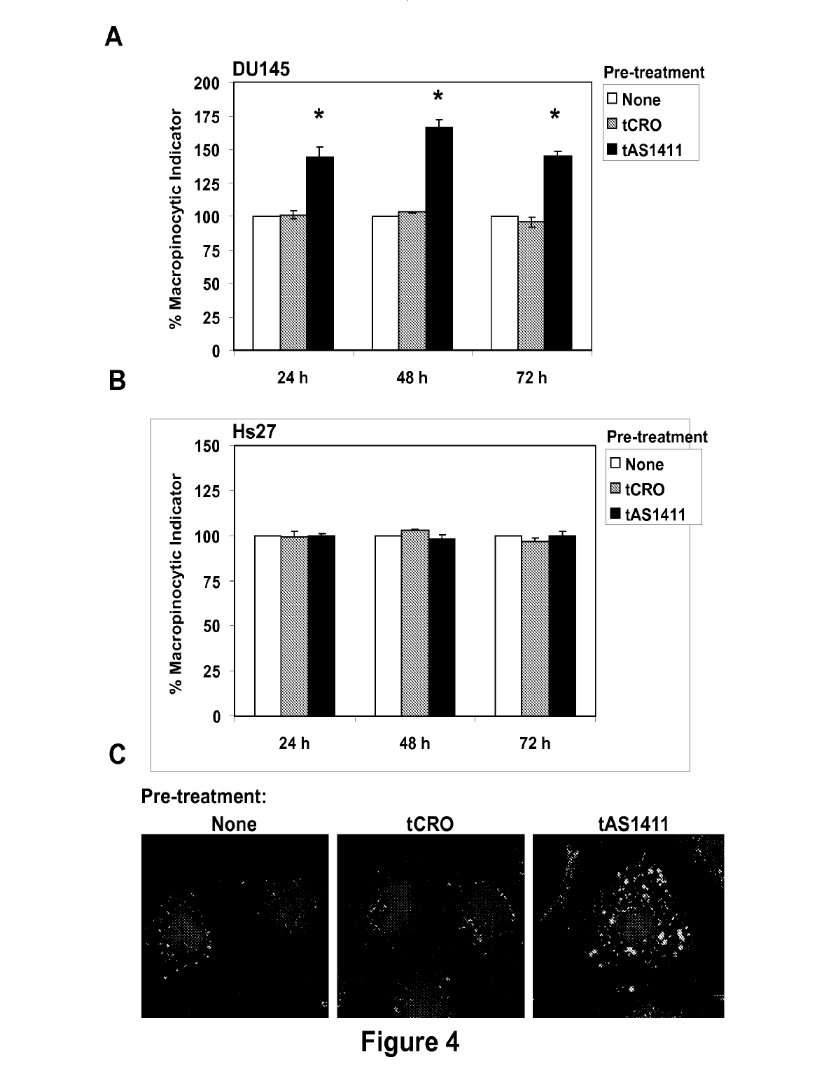

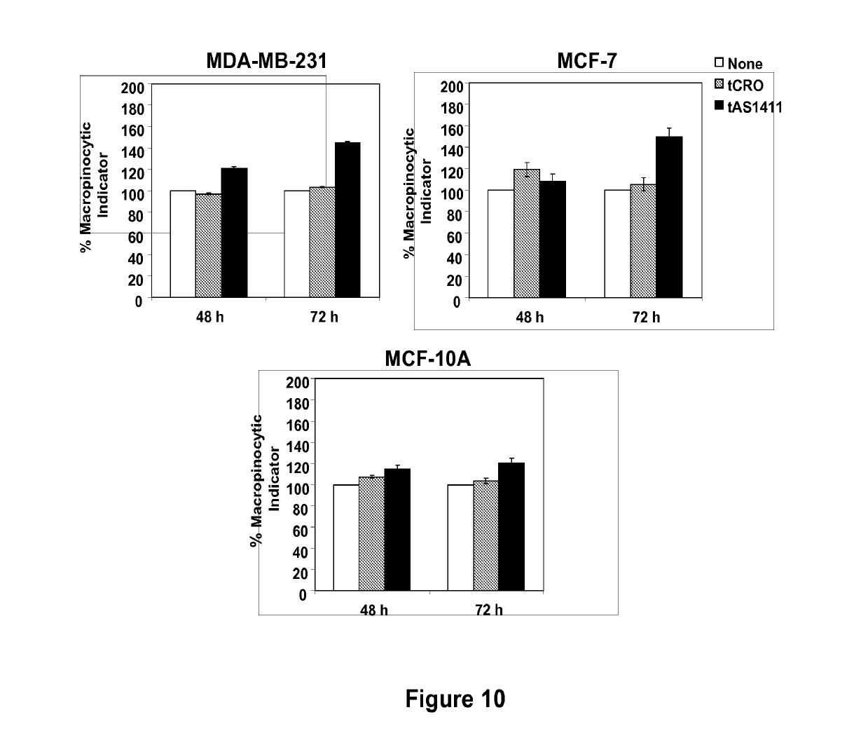

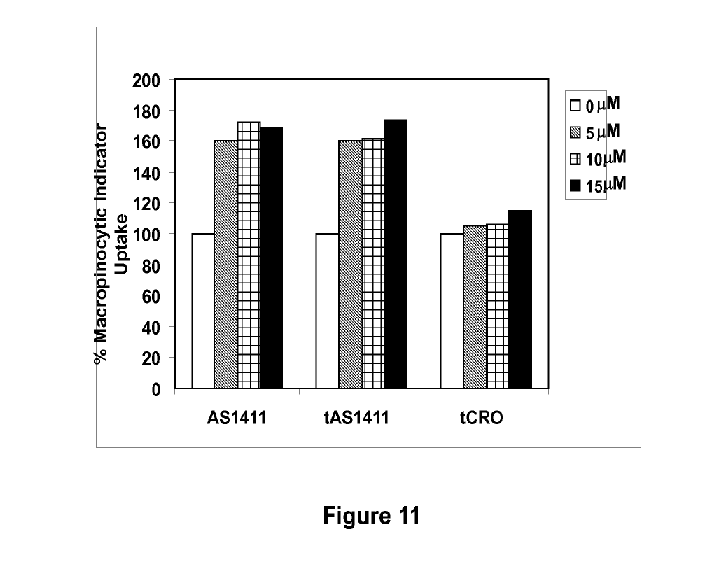

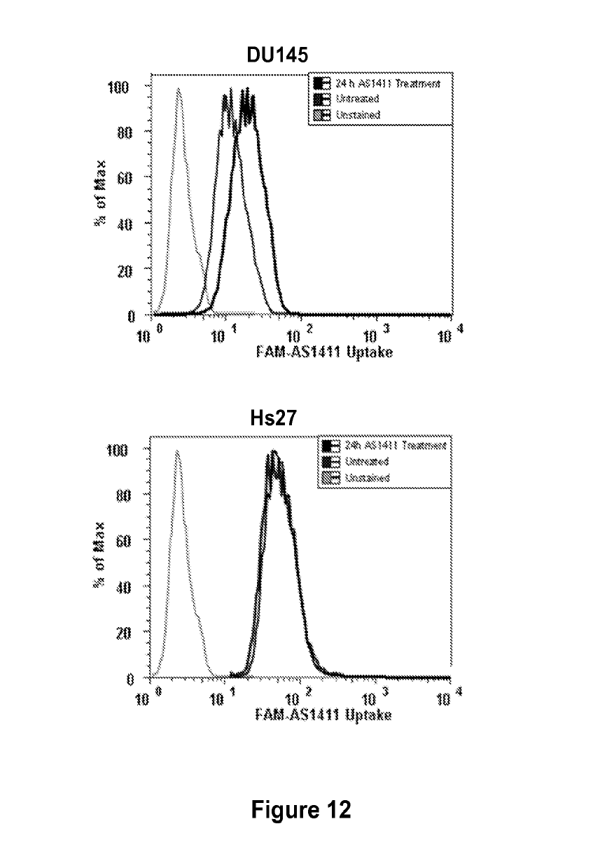

AS1411 causes a change in cancer cell morphology that is characterized by vacuolization, irregular nuclei, and swollen cells (Xu et al., 2001, J. Biol. Chem., 276:43221-30). Therefore, the effect of AS1411 on macropinocytosis in DU145 cells and non-malignant Hs27 cells was investigated. Flow cytometry experiments indicated a significant increment in the uptake of the macropinocytic marker, dextran, in DU145 cells treated with tAS1411 (which is FL-AS1411 without the fluorescent label) for 24, 48, or 72 h (FIG. 4A), whereas there was no increase in the Hs27 cells (FIG. 4B). As in all of the flow cytometry experiments, cells were gated to exclude permeable cells, discounting the possibility that this increase was due to cell death. No changes in dextran uptake were observed in DU145 cells treated with the control oligonucleotide, tCRO (FIG. 4A) or with AS1411 for shorter times (1 h, 2 h, and 4 h). The tAS1411 was also able to induce hyperstimulation of macropinocytosis in other cancer cells lines (MCF-7 and MDA-MB-231) and had a much reduced effect in another non-malignant cell type (MCF-10A) (FIG. 10), suggesting that these novel observations may represent a general difference between the response of cancer cells and normal cells. Confocal microscopy confirmed the flow cytometry results and showed that DU145 cells treated with tAS1411 presented a higher dextran uptake confined in large vesicle than the untreated or CRO-treated cells (FIG. 4C). Additional experiments (FIG. 11) confirmed that the 26-mer version of AS1411 was able to induce the same response as tAS1411 (which has three additional thymidines for reasons described in the methods section). One implication of the finding that AS1411 causes hyperstimulation of macropinocytosis is that treatment of AS1411 might actually promote its own internalization by cancer cells. To test this idea, DU145 cells were pre-treated for 24 h with tAS1411, then added FL-AS1411 and evaluated uptake after an additional 2 h using flow cytometry. As predicted, DU145 cells pre-treated with tAS1411, but not those that received control pre-treatments, showed an increase in the uptake of FL-AS1411 in DU145 cells, whereas there was no comparable increase in Hs27 cells (FIG. 12). All of these results indicate that initial AS1411 uptake leads to the stimulation of macropinocytosis, provoking an increase on its own uptake. This idea is not necessarily inconsistent with the time course data shown in FIG. 1A because the fluorescence signal may decrease over time for a number of reasons, including exocytosis of the ligand and fluorescence quenching due to protein binding or environmental factors (the fluorophore used for flow cytometry is particularly sensitive to acidic environments).

### Example 13

### Initial Uptake of AS1411 is Independent of Nucleolin

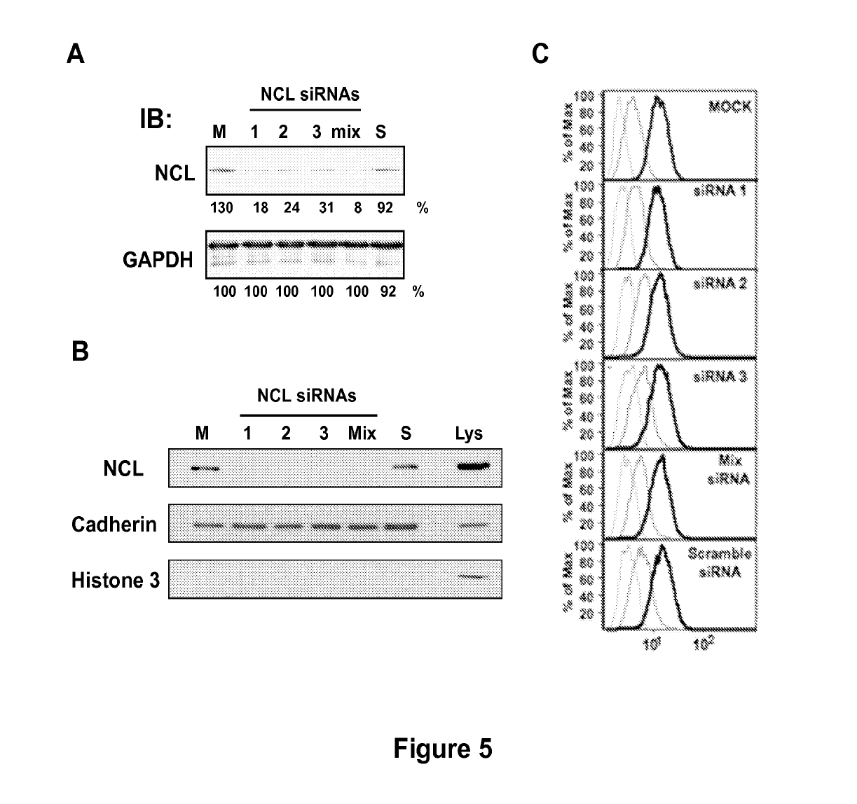

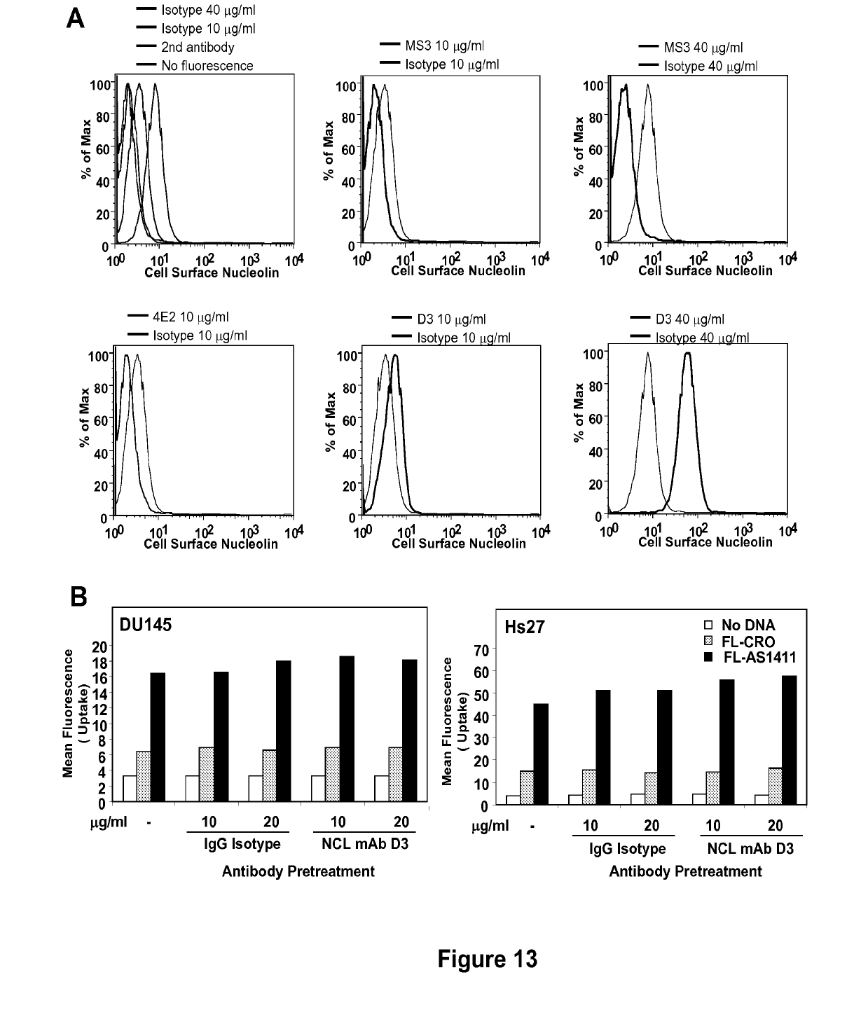

It has been shown previously that nucleolin is the primary molecular target of AS1411 (Bates et al., 2009, Exp. Mol. Pathol., 86:151-64), and it was originally hypothesized that surface nucleolin may serve as a receptor for AS1411. However, the data presented herein are not consistent with that hypothesis because they indicate that uptake occurs, not by classical receptor-mediated endocytosis, but by macropinocytosis. Therefore, the role nucleolin plays in AS1411 uptake was evaluated. The effect of anti-nucleolin mAbs on uptake of FL-AS1411 was first assessed after 2 h incubation using flow cytometry and it was found that none of the anti-nucleolin mAbs tested affected uptake of FL-AS1411 (FIG. 13). Next, similar experiments were carried out using a siRNA approach to knockdown the expression of nucleolin Immunoblot analyses of total DU145 cell lysates using anti-nucleolin antibody showed that expression of total nucleolin could be reduced by more than 80% in cells transfected with nucleolin siRNAs compared with control-transfected cells (FIG. 5A). It was also confirmed that these siRNAs could effectively knockdown the cell surface form of nucleolin (FIG. 5B), using techniques described above. The transfected DU145 cells were next used to assess the uptake of FL-AS1411 after 2 h by flow cytometry analysis (FIG. 5C) and found that knockdown of nucleolin had no effect on FL-AS1411 uptake under these conditions (FIG. 5C).

### Example 14

### Nucleolin Regulates AS1411-Induced Stimulation of Macropinocytosis

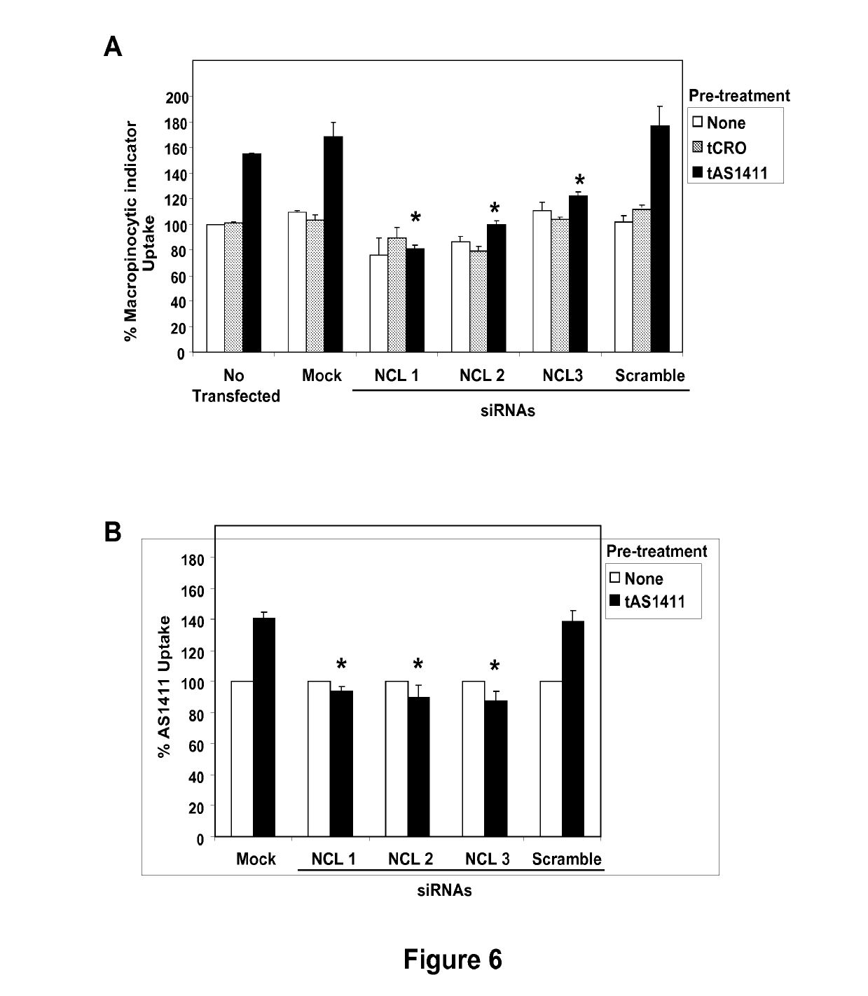

The results shown in FIG. 4 suggest that the induction of macropinocytosis may be an important component of AS1411 activity. Therefore, it was also determined whether nucleolin knockdown affects the tAS1411-mediated stimulation of macropinocytosis observed in DU145 cells. As shown in FIG. 6A, inhibition of nucleolin expression by specific siRNAs had only a marginal effect on the baseline macropinocytosis, but caused a significant decrease in AS1411-induced macropinocytosis, almost completely blocking this process. Accordingly, the tAS1411-induced uptake of FL-AS1411 was also completely blocked in DU145 cells transfected with nucleolin siRNAs (FIG. 6B). These results indicate that, whereas nucleolin does not appear to play a role in the initial macropinocytic uptake of AS1411, it is essential for the AS1411-induced hyperstimulation. Consequently, nucleolin is also essential for the induced uptake of AS1411 that occurs at later time points.

### Example 15

### Additional G-Rich Oligonucleotides and Macropinocytosis

A number of additional G-rich oligonucleotides were obtained and used to evaluate whether or not macropinocytosis was increased in cancer cells using the methodology described herein. For example, the following sequences were used:

Pu27
(SEQ ID NO: 10)
TTATGGGGAGGGTGGGGAGGGTGGGGAAGG
Pu24C
(SEQ ID NO: 11)
TGAGGGTGGCGAGGGTGGGGAAGG
Myc-22
(SEQ ID NO: 12)
TGAGGGTGGGTAGGGTGGGTAA
Myc-1245
(SEQ ID NO: 13)
TGGGGAGGGTTTTTAGGGTGGGGA
Myc-2345
(SEQ ID NO: 14)
TGAGGGTGGGGAGGGTGGGGAA
ckit1
(SEQ ID NO: 15)
CAGAGGGAGGGCGCTGGGAGGAGGGGCTG
ckit2
(SEQ ID NO: 16)
CCCCGGGCGGGCGCGAGGGGAGGGGAGGC
VEGF
(SEQ ID NO: 17)
CCCGGGGCGGGCCGGGGGCGGGGTCCCGGCGGGGCGGAG
HIF-1a
(SEQ ID NO: 18)
GCGAGGGCGGGGGAGAGGGGAGGGGCGCG
bcl-2
(SEQ ID NO: 19)
GTCGGGGCGAGGGCGGGGGAAGGAGGGCGCGGGCGGGGA
k-ras
(SEQ ID NO: 20)
GGGAGGGAGGGAAGGAGGGAGGGAGGGA
Rb
(SEQ ID NO: 21)
CGGGGGGTTTTGGGCGGC
AS1411
(SEQ ID NO: 3)
TTTGGTGGTGGTGGTTGTGGTGGTGGTGG
CRO
(SEQ ID NO: 6)
TTTCCTCCTCCTCCTTCTCCTCCTCCTCC

A small number of the G-rich sequences evaluated did not stimulate macropinocytosis, but most of the G-rich oligonucleotides used increased macropinocytosis in DU145 prostate cancer cells from 10% over the untreated control up to 51% over the untreated control cells.

In addition, the G-rich oligonucleotides disclosed in Dapic et al. (2003, Nuc. Acids Res.,31:2097 -107; KS-A though KS-I) and the G-rich oligonucleotides (e.g., telomere homologs, GT oligonucleotides, Stat3 binders, Dz13, and triplex oligonucleotides with aptameric effects) disclosed in Bates et al. (2009, Exp. Mol. Path., 86:151-64) and references therein are shown to stimulate macropinocytosis in cancer cells.

### Example 16

### Mechanism By Which AS1411 Causes Cell Death

It has recently been discovered that AS1411 can stimulate macropinocytosis (MP) in cancer cells and this finding was verified by several different methods and in multiple cancer cell lines (Table 1).

TABLE 1

| | | Update by | Stimulate | Cells |
|---|---|---|---|---|
| Cell Line | Description | MP? | MP? | respond? |
| Hs27 | Non-cancer, skin | No | No | No |
| | fibroblasts | | | |
| RWPE1 | Non-cancer, prostate | No | No | No |
| | epithelial | | | |
| BEAS2B | Non-cancer, lung | Low | No | No |
| | epithelial | | | |
| MCF10A | Non-cancer, breast | Low | No | No |
| | epithelial | | | |
| CHO | Non-cancer, hamster | No | No | No |
| | ovary | | | |
| A549 | Cancer, non-small cell | Yes | Yes | Yes |
| | lung | | | |
| DU145 | Cancer, prostate | Yes | Yes | Yes |
| MCF7 | Cancer, breast | Yes | Yes | Yes |
| MDA-MB-231 | Cancer, breast | Yes | Yes | Yes |
| RCC4 | Cancer, renal cell | Yes | Yes | Yes |
| SK-N-DZ | Cancer, neuroblastoma | N.D. | Yes | Yes |

DU145 s.c. xenografts are established on the rear flanks of 6-week old male athymic (nu/nu) mice. When the tumors reach approximately 400 mm³, mice are treated by i.p. injections of AS1411 twice daily for 7 days at a dose of 10 mg/kg/dose. Following euthanasia of mice, tumors are excised, fixed in formalin, and processed for transmission electron microscopy (TEM), standard histochemical staining (H&E, PAS) and immunohistochemistry. Tumor cell morphology is evaluated, the presence of macrophages and other immune cells is assessed, and markers of various forms are stained for cell death and molecules that are involved in MP and methuosis (Ras, Rac1, etc.). To visualize MP in vivo, a protocol similar to that first described by Lencer et al. (1990, Am. J. Physiol., 258:C309-17) is used. Briefly, this involves intravenous infusion of fluorophore-labeled 10 kDa fixable dextran (a fluid phase marker, which was used for the cell-based studies), followed by in vivo fixation by perfusion with a paraformaldehyde/lysine/periodate solution. Post-mortem tissues are flash frozen and cut into semi-thin sections using a microtome. Specimens then are observed by fluorescence microscopy and TEM (following photochemical reaction of p-diaminobenzidine, catalyzed by the fluorophore). To examine the role of MP in initial uptake and antitumor activity of AS1411, amiloride, a Na+/H+ exchanger inhibitor that blocks MP will be utilized. Amiloride is FDA-approved for human use as a diuretic and has been used extensively in experimental animals, including as an in vivo inhibitor of MP. To examine initial uptake, mice are co-injected with 10 mg/kg fluorophore-labeled AS1411 plus 150 μg amiloride, then mice are euthanized after 2 h and tumors excised, fixed and examined by fluorescence microscopy. As a control for specificity, the effect of amiloride also is assessed on uptake of fluorophore-labeled transferrin (which is internalized by receptor-mediated endocytosis and not MP) using in vivo dosing that has been described for other purposes (Sparks et al., 1983, Cancer Res., 43:73-7). It will be examined whether daily amiloride co-treatment can block AS1411 anti-tumor activity (assessed by tumor volume) using proper controls to account for any effects of amiloride alone on tumor growth.

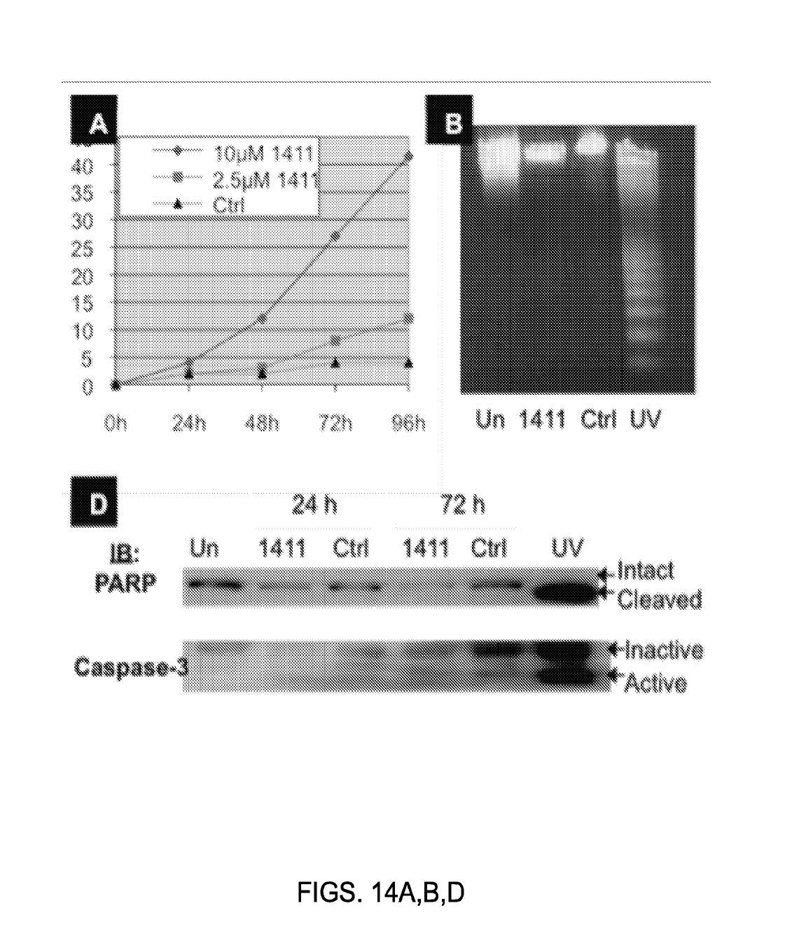

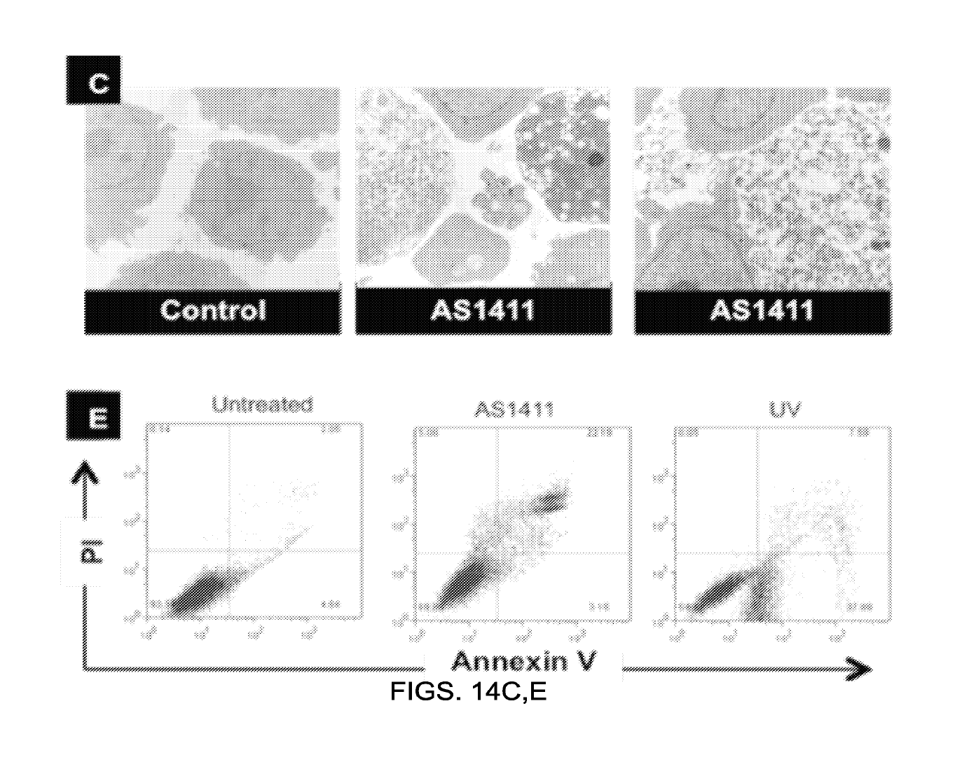

A lack of apoptosis in DU145 cells treated with AS1411 is confirmed using the methods outlined below for U937 cells (FIG. 14). Markers of autophagy are then evaluated. Experiments include Western blots to detect expression of LC3-II and Beclin 1, transfection of cells with LC3-GFP to assess LC3-positive vacuoles, and examination of autophagic flux (levels of LC3-II and p62 in the absence or presence of bafilomycin A). Next, it will be determined whether additional inhibitors of autophagy can affect AS1411 activity. These will include siRNAs to knock down beclin, Atg5, LC3, and Ulk1. Rapamycin treatment will be used as a positive control for autophagy induction. To determine whether ER stress is induced by AS1411, levels of PDI, calreticulin, calnexin, and Nrf2 expression are examined by Western blotting. Calphostin-C is used as a positive control for cell death involving ER stress. The role of proteases that mediate necrosis, including calpains and cathepsins is examined. Methods for all of these assays are widely used and well established. In addition to ruling out other mechanisms of cell death, the timing, dose-dependence, and ultrastuctural features of AS1411-induced macropinocytosis, vacuolization, and cell death in DU145 cells is further characterized using live cell videomicroscopy and electron microscopy.

As described above, it is known that AS1411 can induce an unusual form of cell death in cancer cells. It was previously shown that G-rich oligonuclotides could induce cell death selectively in cancer cells compared to non-malignant cells, but it was noted that the morphology of the cells was inconsistent with death by apoptosis. The timing of cell death was also quite unusual, with continuous exposure (at 10 μM AS1411) for 7 days or more required to cause complete cell death for most cancer cells tested. Interestingly, this time course is similar to that seen during induction of methuosis by ectopic Ras expression. However, cell death was also dose-dependent and occurred within hours in DU145 cells treated with 40 μM AS1411. Based on the various published reports, the cell death mechanism in U937 leukemia cells was investigated, and it was confirmed that cell death was not by apoptosis (FIG. 14 A-D). In contrast to apoptotic cell death (which was induced here by UV irradiation), AS1411 does not induce DNA laddering, PARP cleavage, or Caspase-3 activation (FIG. 14). Also, pre-incubation with caspase inhibitors (zVAD-fmk, zDEVD-fmk, zIETD-fmk, zLEHD-fmk)) or a PARP inhibitor (3-aminobenzamide) did not inhibit AS1411-induced cell death. The electron micrographs of AS1411-treated cells showed necrosis-like cell death characterized by large amounts of cellular debris and degenerating cells. Those cells that had intact plasma membranes showed no signs of apoptosis (e.g. pyknosis, blebbing, or shrinkage), but, instead, were greatly enlarged with swollen organelles, irregular nuclei, large numbers of ribosomes, and extensive vacuoles. In the U937 cells, AS1411 inhibited DNA replication and cell division, but protein synthesis was not inhibited, perhaps suggesting a loss of coordination between cell growth and division. Although the same detailed studies of cell death have not been carried out in other cancer cell lines, it is consistently seen that AS1411-responsive cells die with a characteristic morphology (enlarged and vacuolated cells) without evidence of apoptosis. In addition, flow cytometry studies to assess cell death in several cell lines showed that AS1411 causes cells to appear in the Annexin V-positive/propidium iodide (PI)-positive quadrant (indicative of necrosis), rather than the Annexin V-positive/PI-negative quadrant (apoptosis).

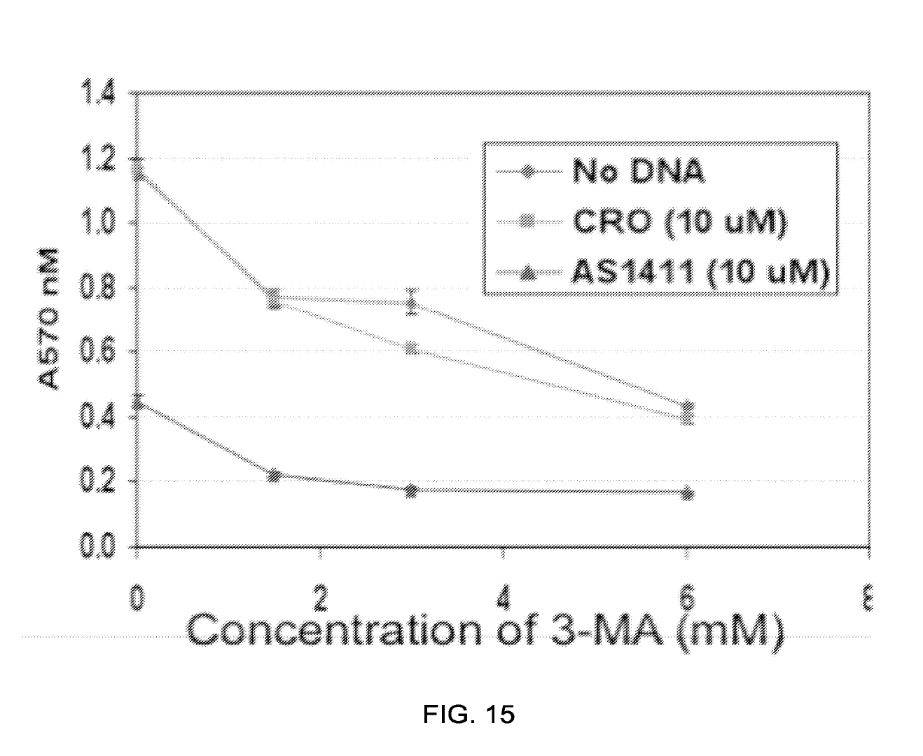

It also appears unlikely that AS1411-induced cell death is due to autophagy. Not only is the ultrastructural morphology quite different (the vacuoles in AS1411-treated cells have single membranes and do not usually contain organelles), but also the autophagy inhibitor, 3-methyladenine (3-MA), did not inhibit AS1411 activity (FIG. 15). Further evidence that supports the idea that AS1411 can induce methuosis comes from the similarity between the appearance of cells treated with AS1411 and published images of glioblastoma cells undergoing Ras-induced methuosis.

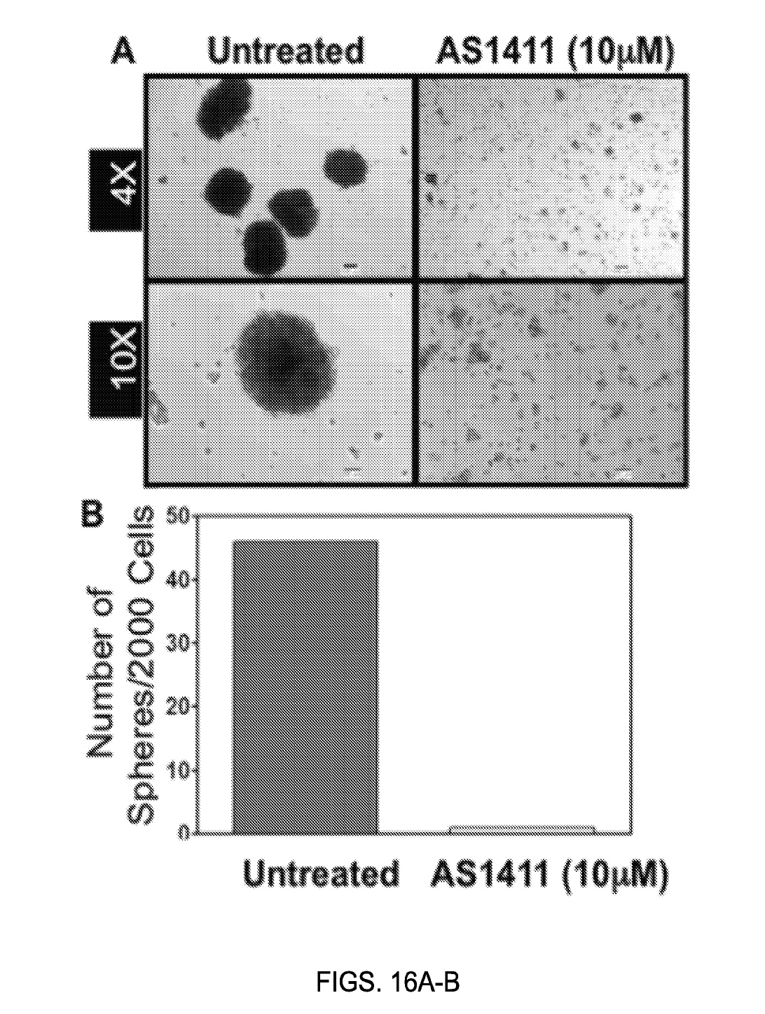

Protocols were recently established for the growth of DU145 cells as spheroids using low adherence plates and specialized medium, and experiments showed that AS1411 can cause disintegration of spheroids (FIG. 16).

### Example 17

### Expression of EGFR, Ras, and Rac

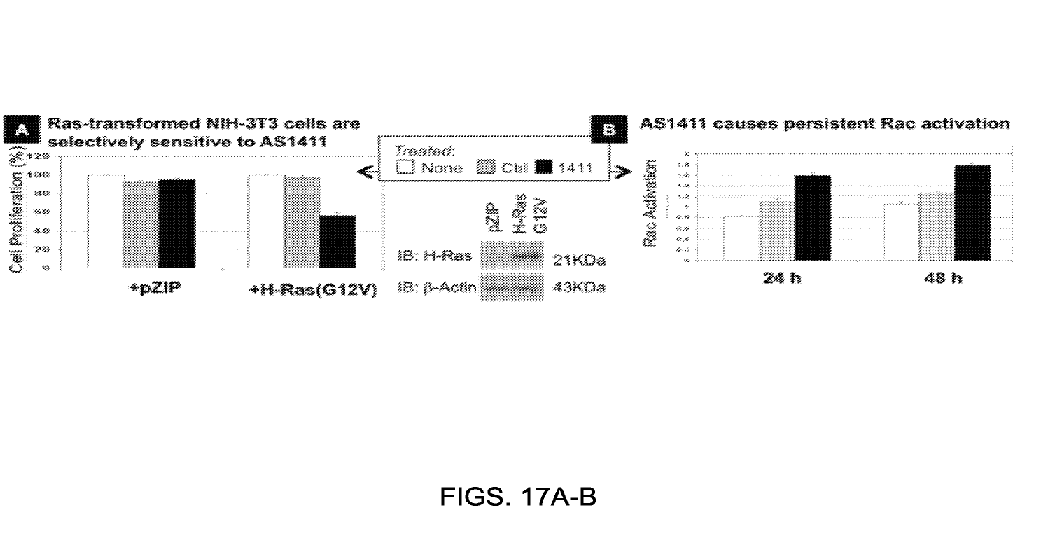

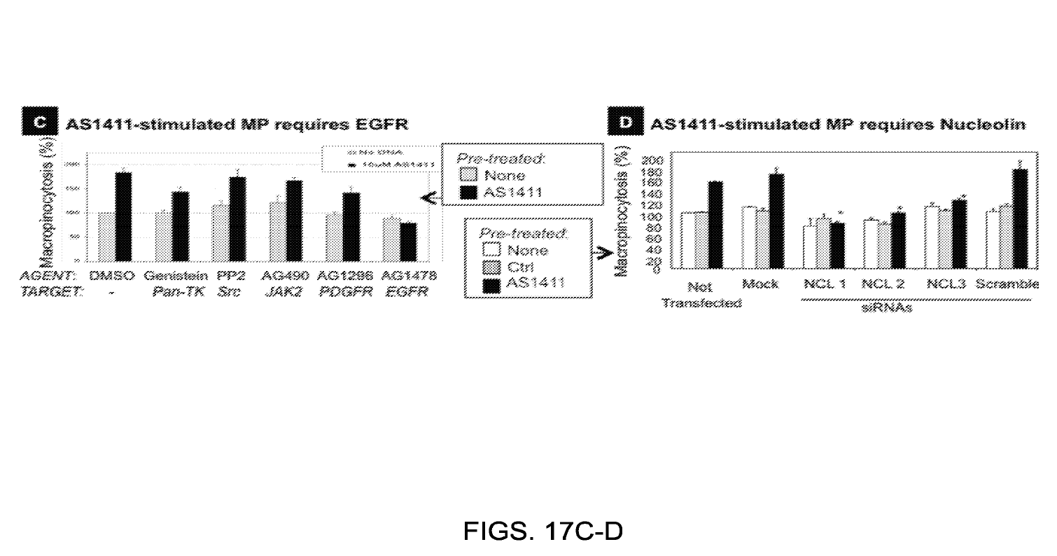

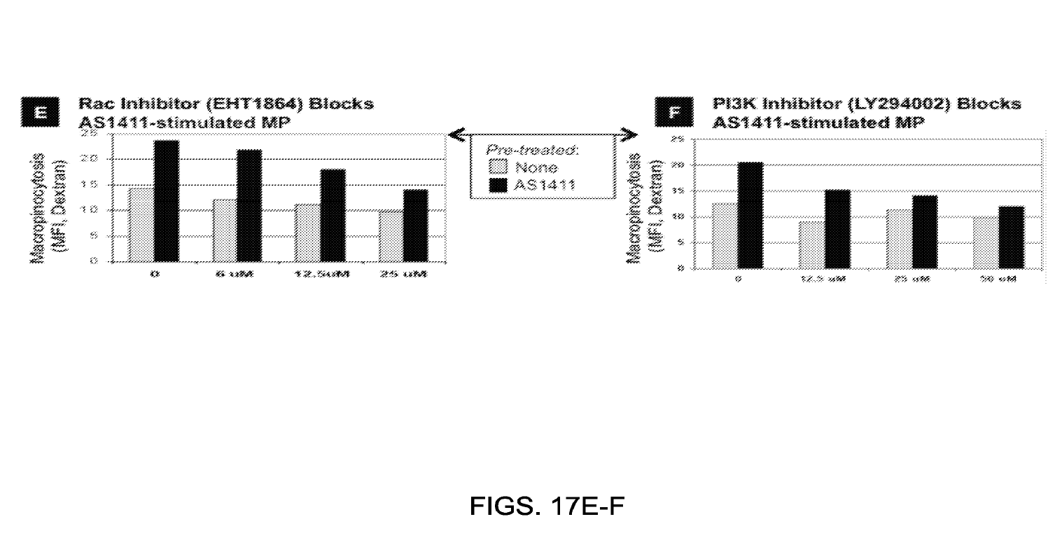

Expression of EGFR, Ras, and Rac pathways was evaluated at various times following treatment of DU145 cells with AS1411 or controls. Total protein levels for EGFR, H/K/N-Ras, and Rac 1/2/3 is determined. Constitutive and EGF-stimulated activation of EGFR receptor is examined by looking at receptor phosphorylation, dimerization and degradation in the absence or presence of AS1411. Ras and Rac activation is assessed using binding domain pull-downs (Raf-RBD and PAK-PBD) followed by Western blotting for various isoforms. Activation of downstream pathways is determined by Western blotting for phosphorylated forms of ERK, Akt, and p38MAPK. Methods for all of these assays are well established and routinely used. For any of the downstream pathways that are activated, it will also be determined whether or not they are essential for AS1411 activity by using siRNA knockdown and pharmacological inhibitors. AS1411 activity is evaluated based on the stimulation of MP, percentage of cells with vacuolization, and anti-proliferative activity (where possible, because persistent inhibition of some targets will be toxic). Next, the effects of constitutively active (CA) or dominant negative (DN) forms of Ras and Rac1 are examined on AS1411-induced MP and cell vacuolization (and, where possible, cell death). In addition, the EGFR-dependence of AS1411-stimulared MP is confirmed using siRNAs to knockdown EGFR expression. To investigate possible roles of nucleolin in mediating upstream events during the AS1411-induced activation of Rac (FIG. 17), the effects of AS1411 on the interactions between nucleolin and EGFR and K-Ras is determined. The presence of nucleophosmin (NPM) in the precipitated complexes also is assessed.

AS1411-induced MPsomes is characterized and it is confirmed that they undergo abnormal trafficking, as observed during Ras-induced methuosis. Evidence that AS1411-induced MPsomes avoid lysosomal fusion also is relevant. Co-localization of AS1411-induced MPsomes is evaluated with markers for various endosomes and lysosomes (e.g., EEA1, LAMP1, Lysotracker Red, Magic Red, acridine orange, Rab5, Rab7). Changes in lipid composition during trafficking of the AS1411-induced vesicles is probed by expression of GFP-2xFYVE, which specifically binds PtdIns(3)P. The studies for MPsome trafficking are carried out in live cells and are tracked over time using time-lapse video microscopy (both standard and confocal). Additionally, the role of Arf6 and GIT1 in mediating AS1411 effects is examined. These factors lie downstream of Rac, are important for MPsome trafficking, and were recently found to play a role in methuosis.

Finally, it will be determined whether the AS1411-induced molecular changes found in cultured cells also occur in vivo. This is achieved by immunohistochemical staining of AS1411-treated tumors to detect altered protein levels or localization.

### Example 18

### Delivery of siRNA

The ability of AS1411 pre-treatment to improve delivery and activity of molecules that cannot enter cells by passive diffusion is evaluated. These will include siRNAs to polo-like kinase (PLK1), a DNA plasmid encoding the luciferase reporter gene, an antibody to PLK1, phalloidin (a cell-impermeable toxin targeting actin), and gelonin (a cell impermeable toxin that inactivates ribosomes). These examples were chosen because methods for their use (including dosing) have been previously reported and because, in some cases, they have demonstrated activity against prostate cancer cells when delivered in a targeted fashion. Delivery is monitored by flow cytometry and confocal microscopy using fluorescently tagged molecules (siRNAs, plasmids), by indirect immunofluorescence (gelonin, PLK1 antibody), or by the intrinsic fluorescence of the molecule (phalloidin). Functional outputs include target knockdown, luciferase activity, and antiproliferative effects measured using the MTT assay after 4 and 7 days of treatment. For the last assay, the combination index for agents (added at the same time or 48 h after AS1411) is determined to identify any synergistic or additive effects. Effects on non-malignant cells, including dendritic cells and macrophages, are assessed. Similar methods are used to test AS1411 in combination with agents that activate MP and Rac. These include EGF, TAT protein transduction domain (a cell penetrating peptide), caffeine, hyaluronan, methamphetamine, and FTY720 (a sphingosine-1-phosphate receptor agonist). These were chosen because methods for their use are well established and their ability to stimulate MP or Rac activation has been well documented. In addition, many of these are FDA-approved for human use in non-cancer indications (EGF, methamphetamine, hyaluronan, FTY720, caffeine).

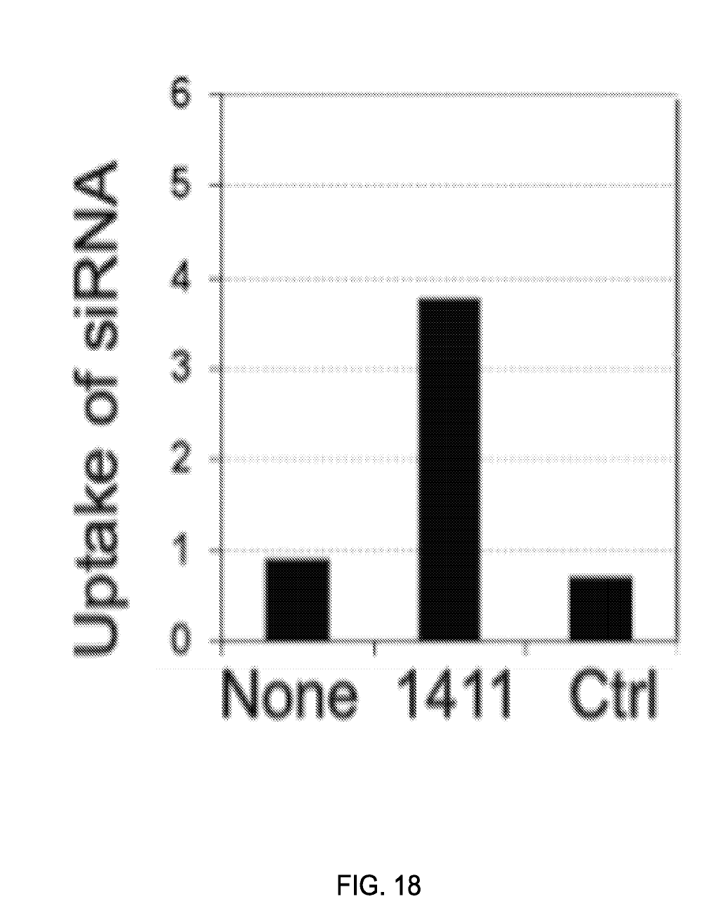

Pre-treatment of cancer cells with AS1411 is used to increase cellular delivery of molecules that do not easily cross the plasma membrane. Furthermore, due to the unique properties of MPsomes, delivery by MP leads to increased functional activity. Thus, treatment with AS1411, followed by administration of an anticancer siRNA, for example, leads to a synergistic increase in anticancer effects without harming normal cells. Another strategy to potentiate the effects of AS1411 is to combine it with agents that promote MP and activation of Rac. This leads to increased macropinocytic uptake of AS1411 and/or enhanced methuosis. It has already been shown that pre-treatment of cancer cells with AS1411 leads to induced uptake of dextran, AS1411, or transferrin (by MP) from the culture medium. In addition, the uptake of fluorescently labeled duplex siRNA in DU145 cells pre-treated with AS1411 (10 μM, 48 h) was examined, and a substantial increase in siRNA delivery was observed (FIG. 18).

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A method of stimulating macropinocytosis in cancer cells, comprising the steps of:
contacting said cancer cells with a G-rich nucleic acid that is capable of forming a quadruplex structure,
thereby stimulating macropinocytosis in said cancer cells.

2. The method of claim 1, wherein the G-rich nucleic acid is between 10 and 50 nucleotides in length and is greater than 25% G nucleotides.

3. The method of claim 1, wherein the G-rich nucleic acid has a sequence shown in SEQ ID NO: 1.

4. The method of claim 1, wherein said cancer cells are selected from the group consisting of prostate cancer, lung cancer, cervical cancer, breast cancer, colon cancer, pancreatic cancer, renal cell carcinoma, ovarian cancer, leukemia, lymphoma, melanoma, glioblastoma, neuroblastoma, sarcoma, and gastric cancer.

5. A method of delivering a therapeutic compound to cancer cells, comprising the steps of:
contacting said cancer cells with a G-rich nucleic acid that is capable of forming a quadruplex structure, and
contacting said cancer cells with a therapeutic compound, wherein said therapeutic compound is taken up by the cancer cells via macropinocytosis.

6. The method of claim 5, wherein the therapeutic compound is a nucleic acid, a peptide, a small molecule, a drug, a chemical, an antibody or a nanoparticle.

7. The method of claim 6, wherein the nucleic acid is antisense RNA, interfering RNA, immunostimulatory oligonucleotides, triple helix oligonucleotides, transcription factor decoy nucleic acids, aptamers, or plasmid DNA

8. The method of claim 5, wherein the G-rich nucleic acid is between 10 and 50 nucleotides in length and is greater than 25% G nucleotides.

9. The method of claim 5, wherein the G-rich nucleic acid has a sequence shown in SEQ ID NO: 1.

10. The method of claim 5, wherein said cancer cells are selected from the group consisting of prostate cancer, lung cancer, cervical cancer, breast cancer, colon cancer, pancreatic cancer, renal cell carcinoma, ovarian cancer, leukemia, lymphoma, melanoma, glioblastoma, neuroblastoma, sarcoma, and gastric cancer.

11. A method of determining whether cancer cells are susceptible or refractory to the antiproliferative effects of a G-rich nucleic acid capable of forming a quadruplex structure, comprising the steps of:
contacting said cancer cells with said G-rich nucleic acid; and
determining whether or not macropinocytosis is increased in said cancer cells contacted with said G-rich nucleic acid relative to cancer cells not contacted with said G-rich nucleic acid,
wherein an increase in macropinocytosis by said cancer cells contacted with said G-rich nucleic acid indicates that said cancer cells are susceptible to treatment with said G-rich nucleic acid, wherein the absence of an increase in macropinocytosis by said cancer cells contacted with said G-rich nucleic acid indicates that said cancer cells are refractory to treatment with said G-rich nucleic acid.

12. The method of claim 11, wherein the G-rich nucleic acid is between 10 and 50 nucleotides in length and is greater than 25% G nucleotides.

13. The method of claim 11, wherein the G-rich nucleic acid has a sequence shown in SEQ ID NO: 1.

14. The method of claim 11, wherein said cancer cells are selected from the group consisting of prostate cancer, lung cancer, cervical cancer, breast cancer, colon cancer, pancreatic cancer, renal cell carcinoma, ovarian cancer, leukemia, lymphoma, melanoma, glioblastoma, neuroblastoma, sarcoma, and gastric cancer.

15. The method of claim 11, wherein said method is performed in vitro with cancer cells obtained from a patient diagnosed with cancer.


SEQUENCE LISTING (21 sequences)

SEQ ID NO: 1 - DNA, 26 bp, Artificial Sequence
  oligonucleotide
ggtggtggtggttgtggtggtggtgg

SEQ ID NO: 2 - DNA, 29 bp, Artificial Sequence
  oligonucleotide
tttggtggtggtggttgtggtggtggtgg

SEQ ID NO: 3 - DNA, 29 bp, Artificial Sequence
  oligonucleotide
tttggtggtggtggttgtggtggtggtgg

SEQ ID NO: 4 - DNA, 29 bp, Artificial Sequence
  oligonucleotide
tttcctcctcctccttctcctcctcctcc

SEQ ID NO: 5 - DNA, 26 bp, Artificial Sequence
  oligonucleotide
cctcctcctccttctcctcctcctcc

SEQ ID NO: 6 - DNA, 29 bp, Artificial Sequence
  oligonucleotide
tttcctcctcctccttctcctcctcctcc

SEQ ID NO: 7 - DNA, 42 bp, Artificial Sequence
  oligonucleotide
ggucgucauaccucagaagttcuucugagguaugacgacctc

SEQ ID NO: 8 - DNA, 42 bp, Artificial Sequence
  oligonucleotide
ggcaaagcauugguagcaattuugcauccaaugcuuugcctc

SEQ ID NO: 9 - DNA, 42 bp, Artificial Sequence
  oligonucleotide
cggugaaauugauggaaauttauuuccaucaauuucaccgtc

SEQ ID NO: 10 - DNA, 30 bp, Artificial Sequence
  oligonucleotide
ttatggggagggtggggagggtggggaagg

SEQ ID NO: 11 - DNA, 24 bp, Artificial Sequence
  oligonucleotide
tgagggtggcgagggtggggaagg

SEQ ID NO: 12 - DNA, 22 bp, Artificial Sequence
  oligonucleotide
tgagggtgggtagggtgggtaa

SEQ ID NO: 13 - DNA, 24 bp, Artificial Sequence
  oligonucleotide
tggggagggtttttagggtgggga

SEQ ID NO: 14 - DNA, 22 bp, Artificial Sequence
  oligonucleotide
tgagggtggggagggtggggaa

SEQ ID NO: 15 - DNA, 29 bp, Artificial Sequence
  oligonucleotide
cagagggagggcgctgggaggaggggctg

SEQ ID NO: 16 - DNA, 29 bp, Artificial Sequence
  oligonucleotide
ccccgggcgggcgcgaggggaggggaggc

SEQ ID NO: 17 - DNA, 39 bp, Artificial Sequence
  oligonucleotide
cccggggcgggccgggggcggggtcccggcggggcggag

SEQ ID NO: 18 - DNA, 29 bp, Artificial Sequence
  oligonucleotide
gcgagggcgggggagaggggaggggcgcg

SEQ ID NO: 19 - DNA, 39 bp, Artificial Sequence
  oligonucleotide
gtcggggcgagggcgggggaaggagggcgcgggcgggga

SEQ ID NO: 20 - DNA, 28 bp, Artificial Sequence
  oligonucleotide
gggagggagggaaggagggagggaggga

SEQ ID NO: 21 - DNA, 18 bp, Artificial Sequence
  oligonucleotide
cggggggttttgggcggc